(19) United States
(12) Patent Application Publication
(10) Pub. No.: US 2018/0340001 A1
(43) Pub. Date: Nov. 29, 2018

(54) Probes for 18F Positron Emission Tomography Imaging

(71) Applicants: The Texas A&M University System, College Station, TX (US); University of North Carolina at Chapel Hill, Chapel Hill, NC (US)

(72) Inventors: Francois P. Gabbai, College Station, TX (US); Boris Vabre, Calgary (CA); Kantapat Chansaenpak, College Station, TX (US); Zibo Li, Chapel Hill, NC (US); Mengzhe Wang, Chapel Hill, NC (US); Hui Wang, Chapel Hill, NC (US)

(21) Appl. No.: 15/957,600

(22) Filed: Apr. 19, 2018

Related U.S. Application Data
(60) Provisional application No. 62/504,093, filed on May 10, 2017.

(51) Int. Cl. C07F 9/6506; C07B 59/00; C07F 9/52; A61K 51/04

(52) U.S. Cl. CPC C07F 9/6506; C07B 59/004; C07F 9/52; A61K 2123/00; A61K 51/0453; C07B 2200/05; A61K 51/0482

(57) ABSTRACT

The present invention contemplates a method for synthesizing [¹⁸F] fluoride complexes suitable for performing radio-labeling reactions to generate [¹⁸F] fluorinated species for use as imaging agents. The present invention further contemplates kits for making [¹⁸F] fluoride complexes suitable for performing radio-labeling reactions to generate [¹⁸F] fluorinated species. The present invention further contemplates a method of using [¹⁸F] fluoride prosthetic group for targeted tissue and disease imaging.

### CROSS-REFERENCE TO RELATED APPLICATIONS

The present application claims the benefit of U.S. Provisional Patent Application No. 62/504,093, filed on May 10, 2017, which is incorporated herein by reference.

### STATEMENT OF GOVERNMENTAL SUPPORT

This invention was made with government support under grant number RP130604 awarded by the Cancer Prevention Research Institute of Texas and under grant number P30-CA016086-35-37 awarded by the National Cancer Institute. The government has certain rights in the invention.

### FIELD OF THE INVENTION

The present invention contemplates a method for synthesizing [¹⁸F] fluoride complexes suitable for performing radio-labeling reactions to generate [¹⁸F] fluorinated species for use as imaging agents. The present invention further contemplates kits for making [¹⁸F] fluoride complexes suitable for performing radio-labeling reactions to generate [¹⁸F] fluorinated species. The present invention further contemplates a method of using a [¹⁸F] fluoride prosthetic group for targeted tissue and disease imaging.

### BACKGROUND OF THE INVENTION

Fluorine-18 [¹⁸F] is the most frequently used radionuclide in positron emission tomography (PET). This is, in part, due to the fact that fluorine is an element that is prevalent in organic molecules as well as easy to introduce directly into a molecule of interest. In addition, the labeling of biomolecules; such as, oligonucleotides, peptides, and proteins with radioisotopes for use in PET imaging is an increasingly important research aspect due to the use of these biomolecules in medical diagnostic or therapeutic applications.

Unfortunately, the direct fluorination of peptides and proteins is difficult since harsh reaction conditions (high temperatures, acidic or basic conditions) that are required to get high radiochemical yields may destroy those biomolecules. Therefore, there is an urgent demand to solve this problem with peptides and proteins generally radio-labeled through a suitable prosthetic group under mild conditions, which may provide vital molecule for medical diagnosis and other research applications.

### SUMMARY OF THE INVENTION

The present invention contemplates a method for synthesizing [¹⁸F] fluoride complexes suitable for performing radio-labeling reactions to generate [¹⁸F] fluorinated species for use as imaging agents. The present invention further contemplates kits for making [¹⁸F] fluoride complexes suitable for performing radio-labeling reactions to generate [¹⁸F] fluorinated species. The present invention further contemplates a method of using a [¹⁸F] fluoride prosthetic group for targeted tissue and disease imaging.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Other objects, advantages, and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

In one embodiment, the invention contemplates a zwitterionic compound containing a formally anionic phosphorus(V) fluoride moiety bound to a N-heterocyclic carbene. In one embodiment, the invention contemplates a zwitterionic compound containing a formally anionic phosphorus(V) fluoride moiety bound to a formally cationic group including an ammonium, an iminium, an anilinium, a phopshoniun, a sulfonium, an arsonium, a stibonium, a selenonium, a telluronium. In one embodiment, the said cationic group comprises organic groups amenable to facile conjugation with biomolecules. In one embodiment the said organic groups amenable to facile conjugation with biomolecules are terminated by a functional group selected from the consisting of an alkyne, an azide, a thiol, a caroxilic acid, an N-succinimde ester, a maleimide, a sulfonate, a triflate, and an amine. In one embodiment, the invention contemplates a zwitterionic compound with the structure:

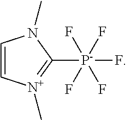

In one embodiment, at least one F is ¹⁸F. In one embodiment, the invention contemplates a zwitterionic compound with the structure:

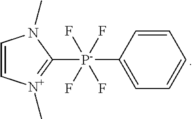 [SMILES: Cn1cc[n+](C)c1[P-](F)(F)(F)(F)c1ccccc1]

In one embodiment, at least one F bound to phosphorus is ¹⁸F.

In one embodiment, the invention contemplates a method for the radiofluorination of a phosphorus(V) fluoride compound, comprising: (a) providing: (i) a phosphorus(V) fluoride compound, and (ii) an [¹⁸F] source, (iii) an isotopic exchange promoter; (b) isotopically exchanging the fluoride in said phosphorus(V) fluoride compound with the [¹⁸F] of said [¹⁸F] source with said isotopic exchange promoter to create an [¹⁸F] phosphorus(V) fluoride compound. In one embodiment, said phosphorus(V) fluoride compound comprises a zwitterionic compound containing a formally anionic phosphorus(V) fluoride moiety bound to a formally cationic group including an ammonium, an iminium, an anilinium, a phopshoniun, a sulfonium, an arsonium, a stibonium, a selenonium, a telluronium. In one embodiment, the said cationic group comprises organic groups amenable to facile conjugation with biomolecules. In one embodiment the said organic groups amenable to facile conjugation with biomolecules are terminated by a functional group selected from the consisting of an alkyne, an azide, a thiol, a caroxilic acid, an N-succinimde ester, a maleimide, a sulfonate, a triflate, and an amine. In one embodiment, said phosphorus(V) fluoride compound is a N-heterocyclic carbene phosphorus(V) fluoride derivative. In one embodiment, said isotopic exchange promoter is SnCl₄. In one embodiment, said [¹⁸F] source is [¹⁸F]-tetra-n-butylammonium fluoride. In one embodiment, said N-heterocyclic carbene phosphorus(V) fluoride derivative is

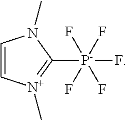

In one embodiment, said N-heterocyclic carbene phosphorus(V) fluoride derivative is

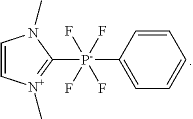 [SMILES: Cn1cc[n+](C)c1[P-](F)(F)(F)(F)c1ccccc1]

In one embodiment, the invention contemplates a method for preparing ¹⁸F-phosphorous-based radiotracers comprising: (a) providing; (i) a phosphorus(V) fluoride compound; (ii) an [¹⁸F] source, (iii) an isotopic exchange promoter; (iv) a biomolecule; (b) isotopically exchanging the fluoride in said phosphorus(V) fluoride compound with the [¹⁸F] of said [¹⁸F] source with said isotopic exchange promoter to create an [¹⁸F] phosphorus(V) fluoride compound; (c) isolating said [¹⁸F] phosphorus(V) fluoride compound; and (d) attaching said [¹⁸F]phosphorus(V) fluoride compound to said biomolecule so as to produce a ¹⁸F-phosphorous-based radiotracer. In one embodiment, said phosphorus(V) fluoride compound comprises a zwitterionic compound containing a formally anionic phosphorus(V) fluoride moiety bound to a formally cationic group including an ammonium, an iminium, an anilinium, a phopshoniun, a sulfonium, an arsonium, a stibonium, a selenonium, a telluronium. In one embodiment, the said cationic group comprises organic groups amenable to facile conjugation with biomolecules. In one embodiment the said organic groups amenable to facile conjugation with biomolecules are terminated by a functional group selected from the consisting of an alkyne, an azide, a thiol, a caroxilic acid, an N-succinimde ester, a maleimide, a sulfonate, a triflate, and an amine. In one embodiment, said phosphorus(V) fluoride compound is a N-heterocyclic carbene phosphorus(V) fluoride derivative. In one embodiment, said isotopic exchange promoter is SnCl₄. In one embodiment, said source of [¹⁸F] is [¹⁸F]-tetra-n-butylammonium fluoride. In one embodiment, said N-heterocyclic carbene phosphorus(V) fluoride derivative is

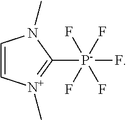

In one embodiment, said N-heterocyclic carbene phosphorus(V) fluoride derivative is

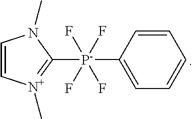 [SMILES: Cn1cc[n+](C)c1[P-](F)(F)(F)(F)c1ccccc1]

In one embodiment, said ¹⁸F-phosphorous-based radiotracer is for Positron Emission Tomography imaging applications. In one embodiment, said biomolecule is selected from the group consisting of protein ligands, antibodies, oligonucleotides, peptides, and proteins. In one embodiment, said biomolecule is a vector. In one embodiment, said vector comprises a moiety that has affinity to a biological target, preferably which accumulates in biological targets due to their biological and/or physiological properties and therefore can be used to visualize biological structures, functions and pathological processes. In one embodiment, said vector is of synthetic or natural origin, and is preferably synthetic. In one embodiment, said vector has the ability to direct the compound to a region of a given disease. Preferably, the vector has affinity for the target, such as a biological receptor, and preferably binds to this. In another aspect, the vector could be an inhibitor, or enzyme substrates and their analogs to reflect enzyme activity. On the one hand the vector should have a high affinity for the receptor, and on the other hand it should “stay” on the receptor as long as necessary. For example, in one embodiment, said the receptors may be located in the vascular system, in the extracellular space, associated with cell membranes or located intracellularly. In one embodiment, said vector can generally be any type of molecule that has affinity for a biological target. In one embodiment, all vectors that can be linked to the N-heterocyclic carbene phosphorus(V) derivative group without losing their affinity to the biological target are relevant. The vector should be physiologically acceptable and should preferably have an acceptable degree of stability. The vector may comprise biomolecules, preferably selected from the group consisting of peptides, peptoids/peptidomimetics and proteins; oligonucleotides, such as oligo-DNA or oligo-RNA fragments; oligosaccharides; lipid-related compounds; hormones; synthetic small drug-like molecules; inhibitors; antibodies and antibody fragments; and derivatives and mimetics thereof. In one embodiment, synthetic peptides of the vector may be obtained by conventional solid phase synthesis, as described by Merrifield employing an automated peptide synthesizer (J. Am. Chem. Soc, 85: 2149 (1963) [1]). In one embodiment, suitable oligonucleotides in a vector comprising oligonucleotides are polymers of ribonucleotides or deoxyribonucleotides comprising between 5 and 100 units, preferably between 10 and 30 units. The oligonucleotides may contain only the five common nitrogen bases of natural nucleic acids, or they may contain non-natural and/or synthetic bases. In one embodiment, suitable oligosaccharides in a vector comprising oligosaccharides are polymers of sugars, containing from three to twenty units, preferably from three to ten units. The constituent sugars are glucose, galactose, mannose, fructose, N-acetylglucosamine, N-acetylgalactosamine or sialic acids, but other sugars, including synthetically modified sugars, may be present. The sugar chains may be linear or branched. In one embodiment, suitable lipid-related compounds in a vector comprising lipid-related compounds are hydrophobic compounds preferably from common building blocks of eukaryotic biological membranes, such as phospholipids, glycolipids or cholesterol. Preferably, they are related to or derived from these compounds. Examples of compounds that are derived from arachidonic acid are prostaglandins and thromboxanes. From phospholipids are derived lysophosphatidylcholine, diacylglycerol and platelet-activating factor, from cholesterol, steroids such as Cortisol, progesterone, estradiol and testosterone. Retinoids also belong in this general class of compounds.

In one embodiment, the invention contemplates a method for preparing ¹⁸F-phosphorous-based radiotracers comprising: (a) providing; (i) a phosphorus(V) fluoride compound; (ii) an [¹⁸F] source, (iii) an isotopic exchange promoter; (iv) a biomolecule; (b) attaching said phosphorus(V) fluoride compound to said biomolecule so as to produce a biomolecule with a phosphorus(V) fluoride compound adduct; and (c) isotopically exchanging the fluoride in said phosphorus(V) fluoride compound with the [¹⁸F] of said [¹⁸F] source with said isotopic exchange promoter to create a biomolecule with [¹⁸F] phosphorus(V) fluoride compound adduct. In one embodiment, said phosphorus(V) fluoride compound comprises a zwitterionic compound containing a formally anionic phosphorus(V) fluoride moiety bound to a formally cationic group including an ammonium, an iminium, an anilinium, a phopshoniun, a sulfonium, an arsonium, a stibonium, a selenonium, a telluronium. In one embodiment, the said cationic group comprises organic groups amenable to facile conjugation with biomolecules. In one embodiment the said organic groups amenable to facile conjugation with biomolecules are terminated by a functional group selected from the consisting of an alkyne, an azide, a thiol, a caroxilic acid, an N-succinimde ester, a maleimide, a sulfonate, a triflate, and an amine. In one embodiment, said phosphorus(V) fluoride compound is a N-heterocyclic carbene phosphorus(V) fluoride derivative. In one embodiment, said isotopic exchange promoter is SnCl₄. In one embodiment, said source of [¹⁸F] is [¹⁸F]-tetra-n-butylammonium fluoride. In one embodiment, said N-heterocyclic carbene phosphorus(V) fluoride derivative is

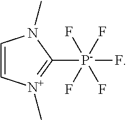

In one embodiment, said N-heterocyclic carbene phosphorus(V) fluoride derivative is

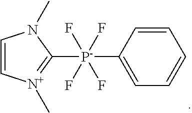 [SMILES: Cn1cc[n+](C)c1[P-](F)(F)(F)(F)c1ccccc1]

In one embodiment, said ¹⁸F-phosphorous-based radiotracer is for Positron Emission Tomography imaging applications. In one embodiment, said biomolecule is selected from the group consisting of protein ligands, antibodies, oligonucleotides, peptides, and proteins. In one embodiment, said biomolecule is a vector. In one embodiment, said vector comprises a moiety that has affinity to a biological target, preferably which accumulates in biological targets due to their biological and/or physiological properties and therefore can be used to visualize biological structures, functions and pathological processes. In one embodiment, said vector is of synthetic or natural origin, and is preferably synthetic. In one embodiment, said vector has the ability to direct the compound to a region of a given disease. Preferably, the vector has affinity for the target, such as a biological receptor, and preferably binds to this. In another aspect, the vector could be an inhibitor, or enzyme substrates and their analogs to reflect enzyme activity. On the one hand the vector should have a high affinity for the receptor, and on the other hand it should “stay” on the receptor as long as necessary. For example, in one embodiment, said the receptors may be located in the vascular system, in the extracellular space, associated with cell membranes or located intracellularly. In one embodiment, said vector can generally be any type of molecule that has affinity for a biological target. In one embodiment, all vectors that can be linked to the N-heterocyclic carbene phosphorus(V) derivative group without losing their affinity to the biological target are relevant. The vector should be physiologically acceptable and should preferably have an acceptable degree of stability. The vector may comprise biomolecules, preferably selected from the group consisting of peptides, peptoids/peptidomimetics and proteins; oligonucleotides, such as oligo-DNA or oligo-RNA fragments; oligosaccharides; lipid-related compounds; hormones; synthetic small drug-like molecules; inhibitors; antibodies and antibody fragments; and derivatives and mimetics thereof. In one embodiment, synthetic peptides of the vector may be obtained by conventional solid phase synthesis, as described by Merrifield employing an automated peptide synthesizer (J. Am. Chem. Soc, 85: 2149 (1963) [1]). In one embodiment, suitable oligonucleotides in a vector comprising oligonucleotides are polymers of ribonucleotides or deoxyribonucleotides comprising between 5 and 100 units, preferably between 10 and 30 units. The oligonucleotides may contain only the five common nitrogen bases of natural nucleic acids, or they may contain non-natural and/or synthetic bases. In one embodiment, suitable oligosaccharides in a vector comprising oligosaccharides are polymers of sugars, containing from three to twenty units, preferably from three to ten units. The constituent sugars are glucose, galactose, mannose, fructose, N-acetylglucosamine, N-acetylgalactosamine or sialic acids, but other sugars, including synthetically modified sugars, may be present. The sugar chains may be linear or branched. In one embodiment, suitable lipid-related compounds in a vector comprising lipid-related compounds are hydrophobic compounds preferably from common building blocks of eukaryotic biological membranes, such as phospholipids, glycolipids or cholesterol. Preferably, they are related to or derived from these compounds. Examples of compounds that are derived from arachidonic acid are prostaglandins and thromboxanes. From phospholipids are derived lysophosphatidylcholine, diacylglycerol and platelet-activating factor; from cholesterol, steroids such as Cortisol, progesterone, estradiol and testosterone. Retinoids also belong in this general class of compounds.

In one embodiment, the invention contemplates a method of imaging the body of a subject comprising: a) providing: i) a subject comprising a tissue, and ii) a [¹⁸F]—N-heterocyclic carbene phosphorus(V) fluoride derivative, b) administering said [¹⁸F]—N-heterocyclic carbene phosphorus(V) fluoride derivative to said subject, and c) imaging said tissue. In one embodiment, said tissues comprises an organ. In one embodiment, said [¹⁸F]—N-heterocyclic carbene phosphorus(V) fluoride derivative is in a form suitable for mammalian administration. In one embodiment, said imaging comprises generating a Positron Emission Tomography image. In one embodiment, said imaging is preferably carried out where the part of said tissue is diseased. In one embodiment, said [¹⁸F]—N-heterocyclic carbene phosphorus(V) fluoride derivative is

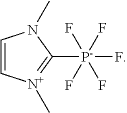

In one embodiment, said [¹⁸F]—N-heterocyclic carbene phosphorus(V) fluoride derivative is

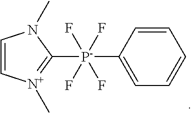 [SMILES: Cn1cc[n+](C)c1[P-](F)(F)(F)(F)c1ccccc1]

In one embodiment, said [¹⁸F]—N-heterocyclic carbene phosphorus(V) fluoride derivative is attached as a prosthetic group to a biomolecule.

In one embodiment, the invention contemplates a method of imaging the human or animal body which comprises generating a Positron Emission Tomography image of at least a part of said body to which a ¹⁸F-phosphorous-based radiotracer composition has distributed. In one embodiment, said ¹⁸F-phosphorous-based radiotracer comprises a radiopharmaceutical composition. In one embodiment, said ¹⁸F-phosphorous-based radiotracer comprises a N-heterocyclic carbene phosphorus(V) fluoride derivative. In one embodiment, said ¹⁸F-phosphorous-based radiotracer is an ¹⁸F-substituted version of the compounds with the structure:

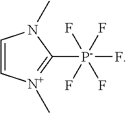

In one embodiment, said ¹⁸F-phosphorous-based radiotracer is an ¹⁸F-substituted version of the compounds with the structure:

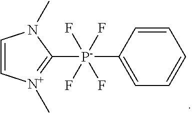 [SMILES: Cn1cc[n+](C)c1[P-](F)(F)(F)(F)c1ccccc1]

In one embodiment, said imaging is preferably carried out where the part of the body is disease state. In one embodiment, said imaging is optionally be carried out repeatedly to monitor the effect of treatment of a human or animal body with a drug, said imaging being effected before and after treatment with said drug, and optionally also during treatment with said drug. Of particular interest is early monitoring of the efficacy of anti-cancer therapy to ensure that malignant growth is controlled before the condition becomes terminal. One example of such therapy monitoring imaging is described by Battle et al J. Nucl. Med., 52(3), 424-430 (2011) [2] and Morrison et al Theranostics, 1, 149-153 (2011) [3]. In one embodiment, said imaging is preferably carried out whereby the radiopharmaceutical composition has been previously administered to the mammalian body. By “previously administered” is meant that the step involving the clinician, wherein the imaging agent is given to the patient e.g. as an intravenous injection, has already been carried out prior to imaging. In one embodiment, said imaging provides a method of diagnosis of the human or animal body.

One embodiment of the present invention contemplates a method for synthesizing a compound with the structure

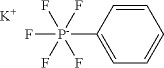 [SMILES: [K+] ; F[P-](F)(F)(F)(F)c1ccccc1]

comprising: a) providing: i) dichlorophenylphosphine; ii) potassium fluoride; and iii) bromine; b) adding bromine to a mixture of potassium fluoride and dichlorophenylphosphine to produce a first reaction mixture; c) stirring said first reaction mixture under such conditions that a reaction occurs; d) evaporating volatile compounds produced by said reaction to produce a residue; e) extracting said residue with a solvent (such as acetonitrile) to produce a first solution, f) filtering said first solution, g) evaporating volatile compounds produced by said reaction to produce a solid product, h) washing said solid product with a solvent (such as diethyl ether), and i) drying said product with the structure

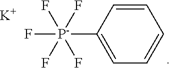 [SMILES: [K+] ; F[P-](F)(F)(F)(F)c1ccccc1]

One embodiment of the present invention contemplates a method for synthesizing a compound with the structure

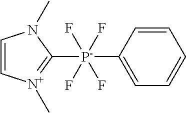 [SMILES: Cn1cc[n+](C)c1[P-](F)(F)(F)(F)c1ccccc1]

comprising: a) providing: i) a compound with the structure

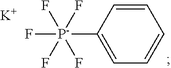 [SMILES: [K+] ; F[P-](F)(F)(F)(F)c1ccccc1]

ii) n-Butyllithium; and iii) dimethylimidazolium iodide; b) adding said n-Butyllithium to a mixture of

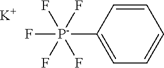 [SMILES: [K+] ; F[P-](F)(F)(F)(F)c1ccccc1]

and dimethylimidazolium iodide to produce a first reaction mixture; c) stirring said first reaction mixture under such conditions that a reaction occurs; d) evaporating volatile compounds produced by said reaction to produce a solid product; e) washing said solid product with water and subsequently ethanol, f) drying said product with the structure

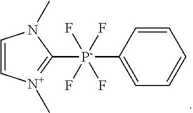 [SMILES: Cn1cc[n+](C)c1[P-](F)(F)(F)(F)c1ccccc1]

In one embodiment, said adding n-Butyllithium to said mixture is under −70° C. In one embodiment, said conditions comprise heating said first reaction mixture to room temperature, then to 65° C.

One embodiment of the present invention contemplates a kit, comprising: a) a first container with a N-heterocyclic carbene phosphorus(V) fluoride derivative, b) a second container with SnCl₄·c) a third container for a [¹⁸F] source, d) a solid-phase extraction cartridge, and e) instructions for use of said kit. In one embodiment, said N-heterocyclic carbene phosphorus(V) fluoride derivative is

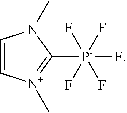

In one embodiment, said N-heterocyclic carbene phosphorus(V) fluoride derivative is

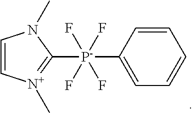 [SMILES: Cn1cc[n+](C)c1[P-](F)(F)(F)(F)c1ccccc1]

In one embodiment, said kit further includes a fourth container with water. In one embodiment, said kit further includes a fifth container with anhydrous solvent. In one embodiment, said solid-phase extraction cartridge has a silica-based bonded phase with strong hydrophobicity and trifunctional bonding chemistry. In one embodiment, said instructions for use comprises: a) obtaining a [¹⁸F] source, b) mixing the contents of said a first container with the contents of said second container with an anhydrous solvent to create a first exchange reaction mixture, c) combining said [¹⁸F] source with said first exchange reaction mixture, d) incubating said first exchange reaction mixture for at least 10 minutes, e) adding water to said reaction mixture to quench said reaction mixture, f) passing the quenched reaction mixture through said solid-phase extraction cartridge, g) passing water through said solid-phase extraction cartridge, and h) eluting the resulting [¹⁸F]N-heterocyclic carbene phosphorus(V) fluoride derivative from said solid-phase extraction cartridge with anhydrous solvent. In one embodiment, said anhydrous solvent is anhydrous acetonitrile. In one embodiment, said [¹⁸F] source comprises [¹⁸F]-tetra-n-butylammonium fluoride. In one embodiment, said incubating occurs at a temperature between room temperature and 100° C. In one embodiment, said kit is designed for use with an automated synthesizer apparatus to prepare the radiopharmaceutical composition.

In addition, the improved compositions of the present inventions can be achieved in shorter preparation times, which minimizes any loss of ¹⁸F (half-life 109 minutes) radioactive content during the preparation and purification steps prior to use. The compositions of the present invention can be obtained using methodology which is amenable to immediate individual preparation or automation on a commercial automated synthesizer apparatus—an advantage over prior art HPLC methods (which cannot be automated in this way). Automation confers improved reproducibility, as well as reduced operator radiation dose.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “solvent” as used herein describes a liquid that serves as a medium for a reaction or a medium for the distribution of components of different phases or extraction of components into said solvent.

As used herein, the term “tetra-n-butylammonium fluoride,” commonly abbreviated to TBAF and n-Bu₄NF, as used herein describes a quaternary ammonium salt with the chemical formula (CH₃CH₂CH₂CH₂)₄N⁺F⁻ with the structure

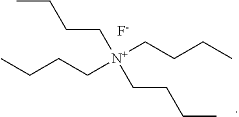

As used herein, the term “tin(IV) chloride” or “tin tetrachloride” or “stannic chloride” as used herein describes an inorganic compound with the formula SnCl₄.

As used herein, the term “N-heterocyclic carbenes” as used herein describes compounds which are a subgroup of a group of compounds called persistent carbenes, a type of carbene demonstrating particular stability. Some examples are described by Herrmann et al.[4] and Hopkinson et al. [5].

As used herein, the term “Prosthetic group”, also referred to as bifunctional labeling agents, are small molecules which contain the radioactive isotope for the radiotracer and are generated to be attached to a target molecule for targeting, for activities such as imaging. In some embodiments, prosthetic groups are generated through introduction of [¹⁸F]fluoride into a small-molecule compound with a second functional group that allows for bioconjugation to a vector or biomolecule under mild conditions.

The term “in vivo imaging” as used herein refers to those techniques that non-invasively produce images of all or part of an internal aspect of a mammalian subject. A preferred imaging technique of the present invention is positron emission tomography (PET).

By the term “biological targeting moiety” (BTM) is meant a compound which, after administration, is taken up selectively or localizes at a particular site of the mammalian body in vivo. Such sites may be implicated in a particular disease state or be indicative of how an organ or metabolic process is functioning.

By the term “amino acid” is meant an L- or D-amino acid, amino acid analogue (eg. naphthylalanine) which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure.

By the term “peptide” is meant a compound comprising two or more amino acids, as defined below, linked by a peptide bond (i.e. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues.

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5). Such compositions lack particulates which could risk causing emboli in vivo, and are formulated so that precipitation does not occur on contact with biological fluids (e.g. blood). Such compositions also contain only biologically compatible excipients, and are preferably isotonic.

The “biocompatible carrier” is a fluid, especially a liquid, in which the imaging agent can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

The imaging agents and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminum). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 50 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may contain additional optional excipients such as: an antimicrobial preservative, pH-adjusting agent, filler, radioprotectant, solubiliser or osmolality adjusting agent. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (i.e. 4-aminobenzoic acid), gentisic acid (i.e. 2,5-dihydroxybenzoic acid) and salts thereof with a biocompatible cation. By the term “biocompatible cation” (Be) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [i.e. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

In one embodiment, preparation of the radiolabeling may be carried out using an automated apparatus. In one embodiment, the biomolecule to which the radiolabel will be attached is created by an automated synthesizer apparatus. By the term “automated synthesizer” is meant an automated module based on the principle of unit operations as described by Satyamurthy ct at Clin. Positr. Imag., 2(5), 233-253 (1999) [6]. The term ‘unit operations’ means that complex processes are reduced to a series of simple operations or reactions, which can be applied to a range of materials. Such automated synthesizers are preferred for the methodof the present invention especially when a radiopharmaceutical composition is desired. They are commercially available from a range of suppliers [Satyamurthy et al, [6]], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapors. The automated synthesizer preferably comprises a cassette. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (e.g. solid phase extraction or SPE). The cassette always comprises a reaction vessel. Such reaction vessels are preferably 0.5 to 10 mL, more preferably 0.5 to 5 mL and most preferably 0.5 to 4 mL in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

Preferred automated synthesizers of the present invention comprise a disposable or single use cassette which comprises all the reagents, reaction vessels and apparatus necessary to carry out the preparation of a given batch of radiofluorinated radiopharmaceutical. The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability, rapid change between production runs; pre-run automated diagnostic checking of the cassette and reagents; automated barcode cross-check of chemical reagents vs. the synthesis to be carried out; reagent traceability; single-use and hence no risk of cross-contamination, tamper and abuse resistance.

The term “protected” refers to the use of a protecting group. By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. For example: amine protecting groups are well known to those skilled in the art and are suitably chosen from: Boc (where Boc is tert-butyloxycarbonyl); Eei (where Eei is ethoxyethylidene); Fmoc (where Fmoc is fluorenylmethoxycarbonyl); trifluoroacetyl; allyloxycarbonyl; Dde [i.e. 1-(4,4-dimethyl-2,6-dioxocyclohexylidene)ethyl] or Npys (i.e. 3-nitro-2-pyridine sulfenyl). The use of further protecting groups are described in Pmtective Groups in Organic Synthesis, 4^{th} Edition, Theorodora W. Greene and Peter G. M. Wuts, (2006) [7].

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows a wide range of known peptide based radiotracers.

FIG. 2 shows N-heterocyclic carbene (NHC) phosphorus(V) fluoride derivatives as contemplated by one embodiment of the present invention.

FIG. 3 shows an exemplary synthesis of target compound 2.

FIG. 4 shows an illustrative ORTEP diagram of 2. Thermal ellipsoids are shown at the 50% probability level. Hydrogen atoms are omitted for clarity. Selected bond distances (Å) and angles (°): P(1)-C(6)=1.839(2); P(1)-F(1)=1.634(1); P(1)-F(2)=1.642(1); P(1)-F(3)=1.631(1); P(1)-F(4)=1.645(1); P(1)-C1)=1.898(2); C(I)—P(1)-(C6)=178.96(6); F(2-P(1)-F(4)=175.75(4); C(6)-P(1)-F(1)=91.76(6).

FIG. 5 shows illustrative ORTEP diagrams of the asymmetric unit (top) and of the packing (bottom) of KPF₅Ph (1). Thermal ellipsoids are shown at the 50% probability level. Hydrogen atoms and K, CH₃CN labels are omitted for clarity. Blue: nitrogen atoms, green: fluorine atoms, purple: potassium atoms, orange: phosphorus atoms, grey: carbon atoms.

FIG. 8 shows exemplary data of kinetic plots for the hydrolysis of 2.

FIG. 6 shows exemplary data of a ¹⁸F{¹H} NMR analysis of an aliquot of the crude reaction mixture for the synthesis of 2 after addition of n-BuLi. The aliquot of the crude mixture is heated at 66° C. and analyzed over time by ¹⁹F {¹H} NMR.

FIG. 7 shows a typical ratio of NHC—PF₄Ph cis and trans (2) isomers during time at 66° C. in THF Ratios are calculated by ¹⁹F NMR integration using BF₃.EtO as internal standard.

FIG. 9 shows an exemplary ¹⁹F NMR spectrum of 1 in D₂O-CD₃CN (8/2 vol) phosphate buffer solution at t=0 and t=5 days.

FIG. 10A shows an exemplary UV-HPLC chromatogram of the acetonitrile (MeCN) portion obtained after radiolabeling of 1 at 100° C.

FIG. 10B shows an exemplary Radio-HPLC chromatogran of the acetonitrile (MeCN) portion obtained after radiolabeling of 1 at 100° C.

FIGS. 11A&B shows an exemplary decay-corrected whole-body microPET-CT images of nude mice from a static scan at 3 h after injection of [¹⁸F]-1. FIG. 11A shows the coronal image, FIG. 11B shows sagittal image.

FIG. 12A shows an exemplary UV-HPLC chromatogram of the acetonitrile (MeCN) portion obtained after radiolabeling of 1 at 100° C.

FIG. 12B shows an exemplary Radio-HPLC chromatogram of the acetonitrile (MeCN) portion obtained after radiolabeling of 1 at 100° C.

FIG. 13 shows an exemplary ¹H NMR spectra of [K][PF₅Ph].

FIG. 14 shows an exemplary ¹³C NMR spectra of [K][PF₅Ph].

FIG. 15 shows an exemplary ³¹P NMR spectra of[K][PF₅Ph].

FIG. 16 shows an exemplary ¹⁹F NMR spectra of [K][PF₅Ph].

FIG. 17 shows an exemplary ¹H NMR spectra of (NHC)PF₄Ph 2.

FIG. 18 shows an exemplary ¹³C NMR spectra of (NHC)PF₄Ph 2.

FIG. 19 shows an exemplary ³¹P NMR spectra of (NHC)PF₄Ph 2.

FIG. 20 shows an exemplary ¹⁹F NMR spectra of (NHC)PF₄Ph 2.

FIG. 21 shows an exemplary HRMS spectra of [K][PF₅Ph].

FIG. 22 shows an exemplary HRMS spectra of (NHC)PF₄Ph 2.

### DETAILED DESCRIPTION OF THE INVENTION

The present invention contemplates a method for synthesizing [¹⁸F] fluoride complexes suitable for performing radio-labeling reactions to generate [¹⁸F] fluorinated species for use as imaging agents. The present invention further contemplates kits for making [¹⁸F] fluoride complexes suitable for performing radio-labeling reactions to generate [¹⁸F] fluorinated species. The present invention further contemplates a method of using a [¹⁸F] fluoride prosthetic group for targeted tissue and disease imaging.

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout the specification may, but do not necessarily, refer to the same embodiment.

Furthermore, the described features, advantages and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

In one embodiment, the invention contemplates a method for the radiofluorination of N-heterocyclic carbene (NHC) phosphorus(V) fluoride adducts. In one embodiment, the IMe-PF5 derivative (IMe=1,3-dimethylimidazol-2-ylidene) undergoes Lewis acid promoted ¹⁸F-¹⁹F isotopic exchange. In one embodiment, the resulting radiofluorinated probe is remarkably resistant to hydrolysis. This is supported by both in vitro and in vivo studies described herein. In the in vitro studies, the release of free fluoride after incubating this probe in aqueous solution (pH 7.5, 80% water/20% acetonitrile) for five days was not observed. In the in vivo studies, free [¹⁸F]-fluoride signal was not observed in a murine model during the full range of the imaging experiment which lasted 3 hours post-injection. Past that point the natural radioactive decay of the fluorine-18 radionuclide led to low signal intensity such that the probe could no longer be studied.

Positron Emission Tomography (PET) is a rapidly growing imaging technique that relies on the use molecular radiotracers containing a positron emitting isotope. To date, a great deal of attention has been devoted to the use of fluorine-18 [¹⁸F], a radionuclide that can be easily generated from [¹⁸O]-water and whose nuclear decay characteristics are ideally suited for applications in PET imaging. One difficulty faced in the synthesis of ¹⁸F-containing molecular radiotracers is the short half-life of the isotope (110 min). It follows that the best methods to access ¹⁸F-containing molecular radiotracers should be fast and preferably carried out in the late stages of the synthesis of the radiopharmaceutical probe. An attractive approach that provides a possible solution to these challenges is based on molecules containing a main group element as a fluoride binding site. In one embodiment, the invention contemplates the preparation of an ¹⁸F-phosphorous-based radiotracer for PET imaging applications. In one embodiment, this new tracer is extremely stable in vivo and has a potential to be used to conjugate with drugs, peptides, proteins, or any diagnostic biomolecules which can be produced as commercial products in pharmaceutical or radiopharmaceutical industries.

McBride and colleagues have also developed many compositions and methods of synthesis and use of ¹⁸F labeled molecules of use, for example, in PET imaging techniques [8-15]. However, in terms of ¹⁸F compounds, undoubtedly, boron-based prosthetic group pioneered by Perrin et al.[16-20] are the most developed. The most versatile example is the alkyne linked zwitterionic ammonium trifluoroborate which can be incorporated in a wide range of peptide based radiotracers. In parallel to these advances, the current invention introduces zwitterionic phosphonium trifluoroborates and carbene-BF₃ adducts which can be conjugated to biomolecule. Following up on these results, there was an attraction to the fluorophilic properties of phosphorus (V) compounds. Indeed, based on computed gas phase fluoride ion affinity data (346 kJ mol−1 for BF₃ and 380 kJ mol−1 for PFS), which show that P(V) species may be more Lewis acidic than boron (III) derivatives, it was determined that phosphorus analogs of the BF₃-carbene might be ideally suited for application in PET. To explore this idea and expand on the limited chemistry of radiofluorinated phosphorus compounds, there was an investigation of the radiofluorination of the N-heterocyclic carbene (NHC) phosphorus(V) fluoride derivatives. In some embodiments, this invention contemplates the synthesis and production of compounds not previously used as radiotracers before. In one embodiment, the current invention contemplates a method of administering a ¹⁸F—PFS-carbene into mice under conditions that demonstrate in vivo stability. In the in vitro studies, the release of free fluoride after incubating this probe in aqueous solution (pH 7.5, 80% water/20% acetonitrile) for five days was not observed. In the in vivo studies, free [18F]-fluoride signal was not observed in a murine model during the full range of the imaging experiment which lasted 3 hours post-injection. Past that point the natural radioactive decay of the fluorine-18 radionuclide led to low signal intensity such that the probe could no longer be studied.

### INTRODUCTION

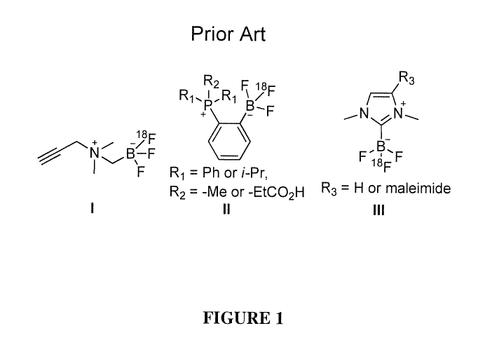

A growing area of radiochemistry is concerned with the discovery of radiolabeled prosthetic groups which, once appended to tissue- or disease-specific biomolecules, provide a modular access to novel Positron Emission Tomography (PET) [21] imaging agents [22-24]. To date, most imaging agents (prosthetic groups) contain a group 13 element (comprising boron (B), aluminum (Al), gallium (Ga), indium (In), thallium (TI)) [22, 25-32] or group 14 element (carbon (C), silicon (Si), germanium (Ge), tin (Sn), lead (Pb), and flerovium (Fl)) which serves as binding site for the fluoride anion [23, 33, 34]. Undoubtedly, boron-based prosthetic groups pioneered by Perrin are the most developed [16-20]. The most versatile example is the alkyne linked zwitterionic ammonium trifluoroborate (I) which can be incorporated in a wide range of peptide based radiotracers (FIG. 1) [35-39]. In parallel to these advances, zwitterionic phosphonium trifluoroborates (II) and carbene-BF₃ adducts (III) were introduced which, like I, can be conjugated to biomolecule [40-42]. Following up on these results, there was attention given to the fluorophilic properties of phosphorus (V) compounds [43-45]. Indeed, based on computed gas phase fluoride ion affinity data (346 kJ mol⁻¹ for BF₃ and 380 kJ mol⁻¹ for PF₅), which show that P(V) species may be more Lewis acidic than boron (EII) derivatives, phosphorus analogs of III appeared suited for application in PET.

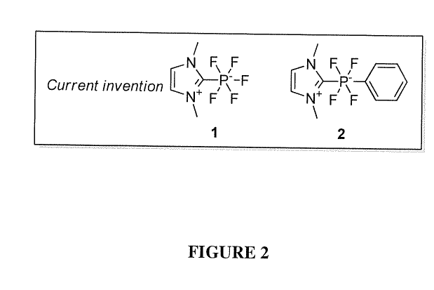

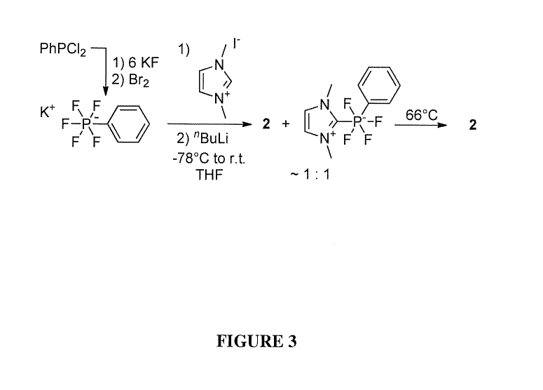

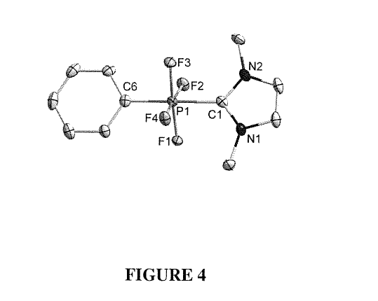

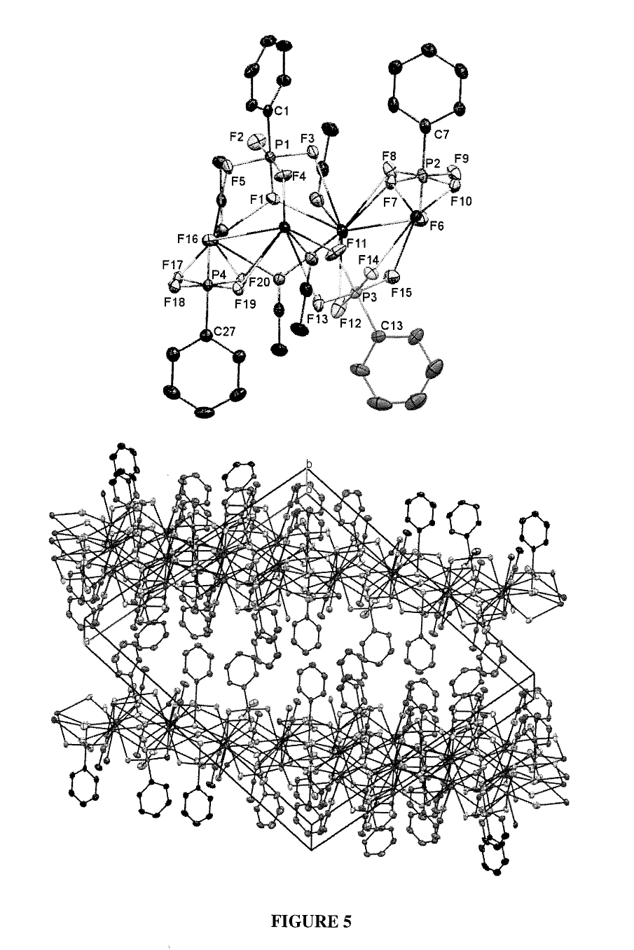

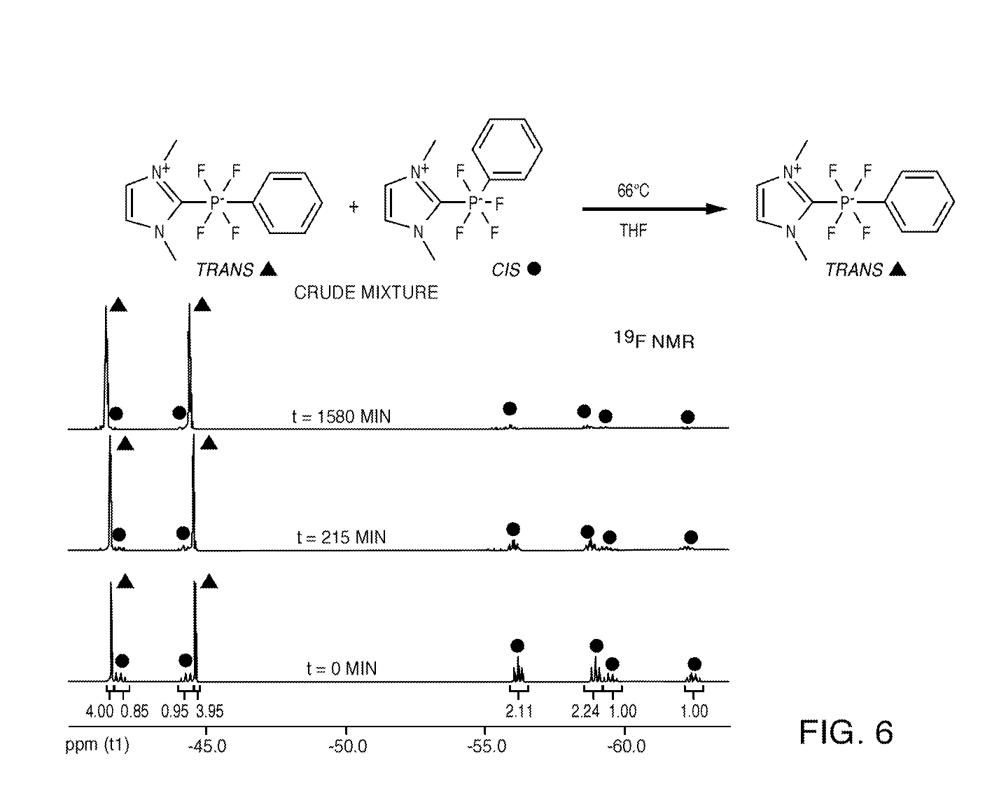

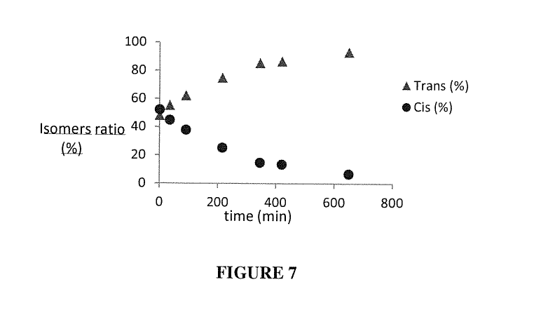

To explore this idea and expand on the limited chemistry of radiofluorinated phosphorus compounds [46, 47], there was an investigation into the radiofluorination of the N-heterocyclic carbene (NHC) phosphorus(V) fluoride derivatives 1 and 2, see FIG. 2. Compound 1 was synthesized as described in the literature. To access compound 2, the potassium salt of the known anion [PF₅Ph]⁺[48] was first synthesized via the “one pot” oxidation of PPhCl₂ using bromine in the presence of KF and structurally characterized (FIG. 4 and FIG. 5). This salt, whose ¹⁹F and ³¹P NMR spectra are consistent with those of previously reported for other [PF₅Ph]⁻ salts [48], was successfully converted into the target compound 2 in 68% yield by addition of n-Bali at −78° C. to a mixture of imidazolium salt and K[PF₅Ph] (FIG. 3). The ¹⁹F NMR analysis of the crude mixture at room temperature after n-BuLi addition shows a doublet (J_{PF}=849 Hz) for 2 at −43.9 ppm and a cis product with an approximate ratio ˜1:1 (Scheme 1). The cis adduct is characterized by three ¹⁹F resonances: a doublet of virtual triplet at −57.6 ppm, (J_{PF}=783 Hz, J_{FF}=40 Hz) and two doublet of doublet of triplet at −43.2 ppm, (J_{PF}=699 Hz, J_{FF}=49 Hz, J_{FF}=40 Hz) and at −61.0 ppm, (J_{FF}=838 Hz, J_{FF}=49 Hz, J_{FF}=40 Hz) with a fluorine integration of 2:1:1 respectively. Heating this mixture at 66° C. for 26 h shows isomerisation of cis product into the trans product 2 (FIG. 6 and FIG. 7). Compound 2 is further characterized by a ³¹P NMR resonance at 141.1 ppm split into a quintet (J_{PF}=849 Hz). The ¹H NMR spectrum shows a characteristic singlet for the methyl substituents while ¹³C NMR shows two doublet of quintet at 150.0 ppm (J_{CF}=43 Hz, J_{CF}=297 Hz) and 159.8 ppm (J_{CF}=71 Hz, J_{CF}=334 Hz) corresponding to the phenyl ipso-carbon and carbene carbon, respectively. These assignments align with those reported for other NHC—PF₄Ph derivatives [49]. The structure of 2 has been confirmed by X-ray diffraction which shows that the carbene-phosphorus C(1)-P(1) distance (1.898(2) A) is only slightly longer than the C(6)-P(1) bond (1.839(2)) involving the phenyl group (FIG. 3).

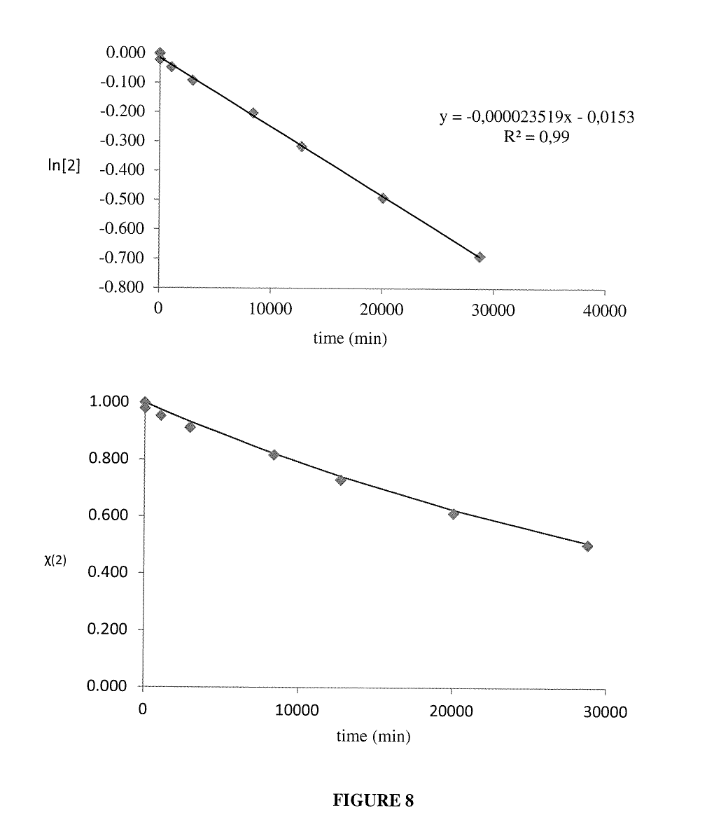

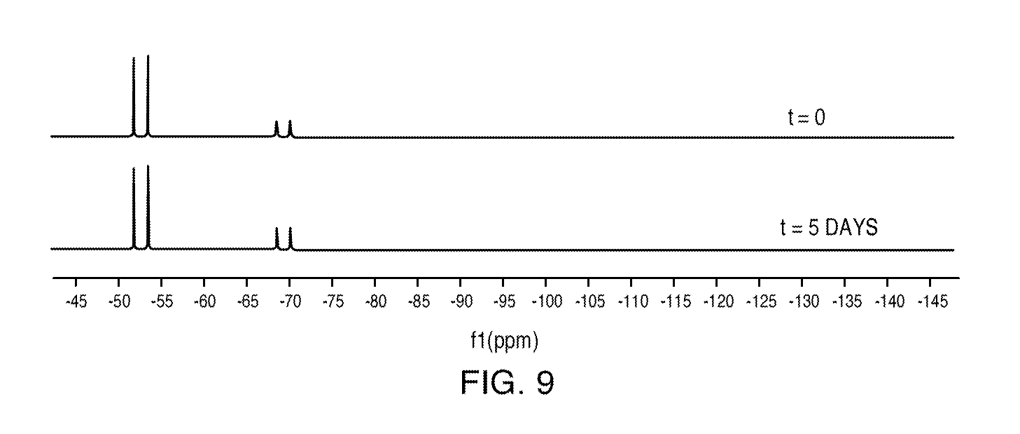

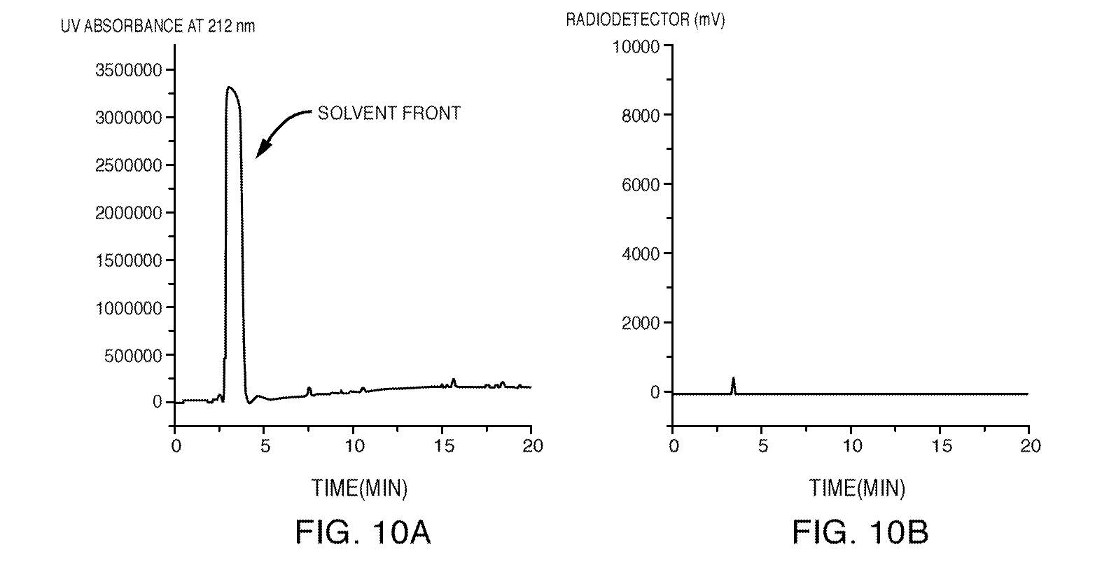

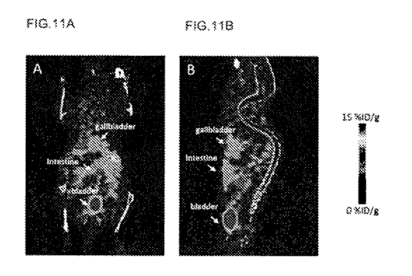

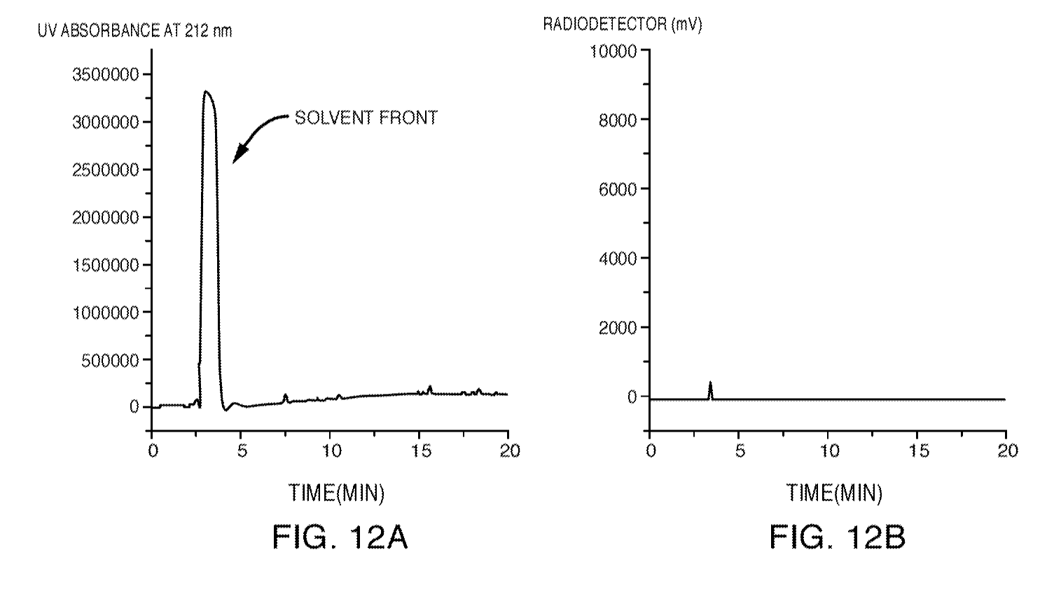

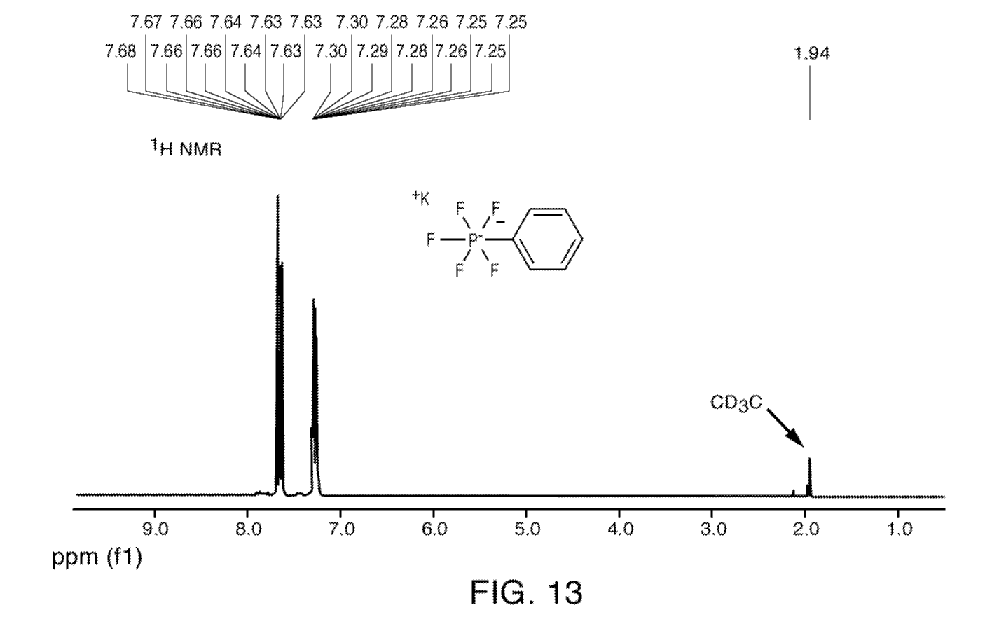

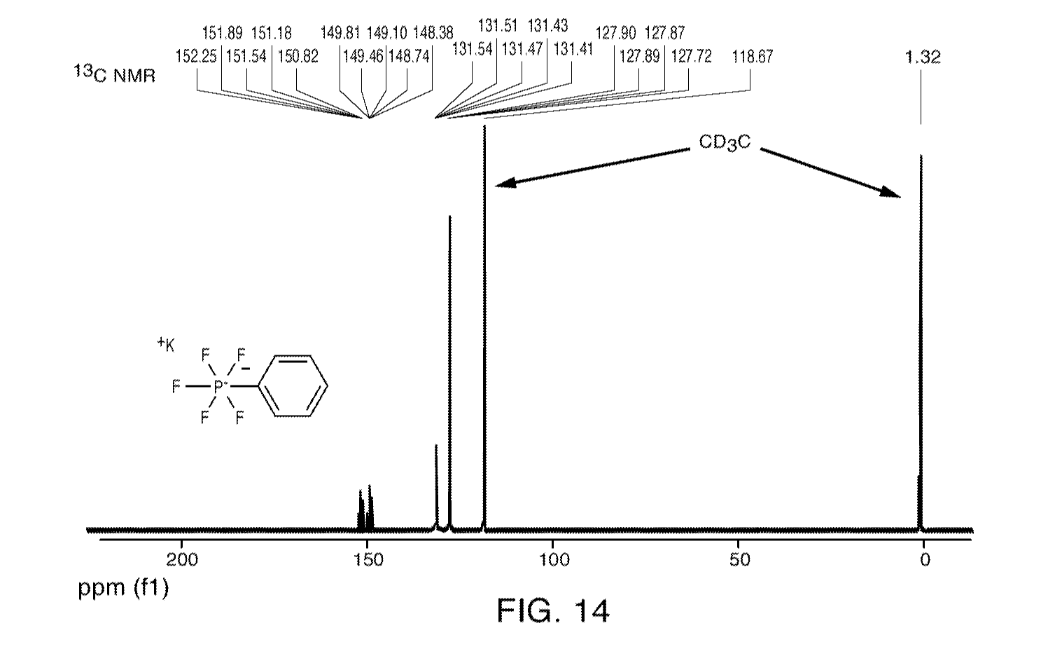

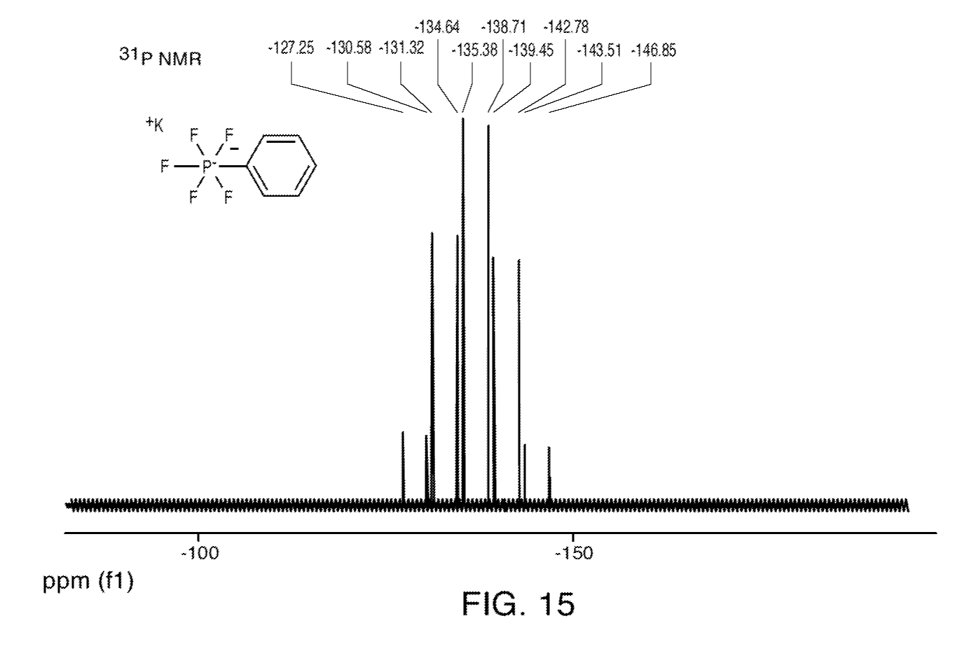

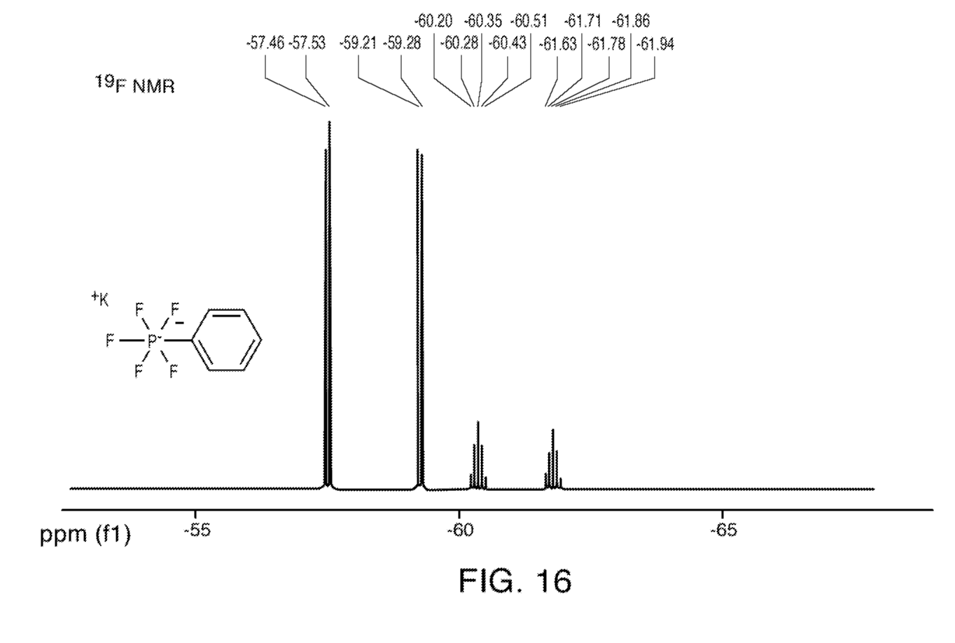

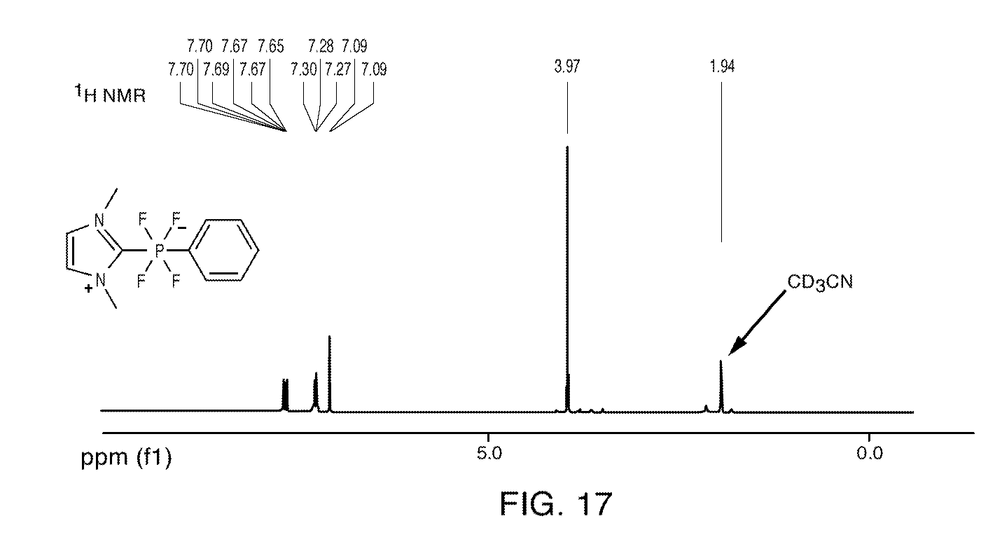

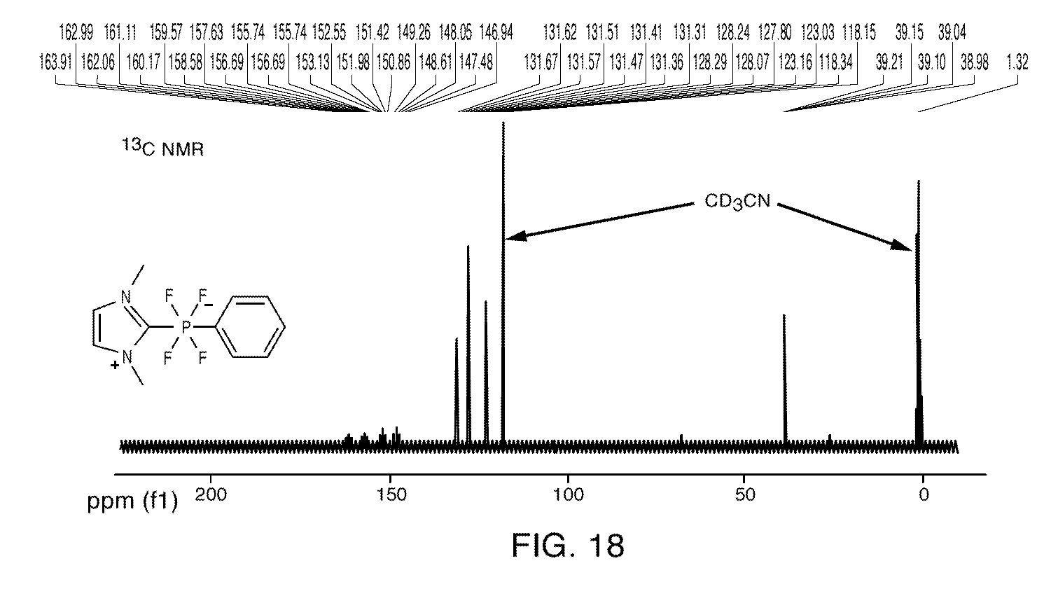

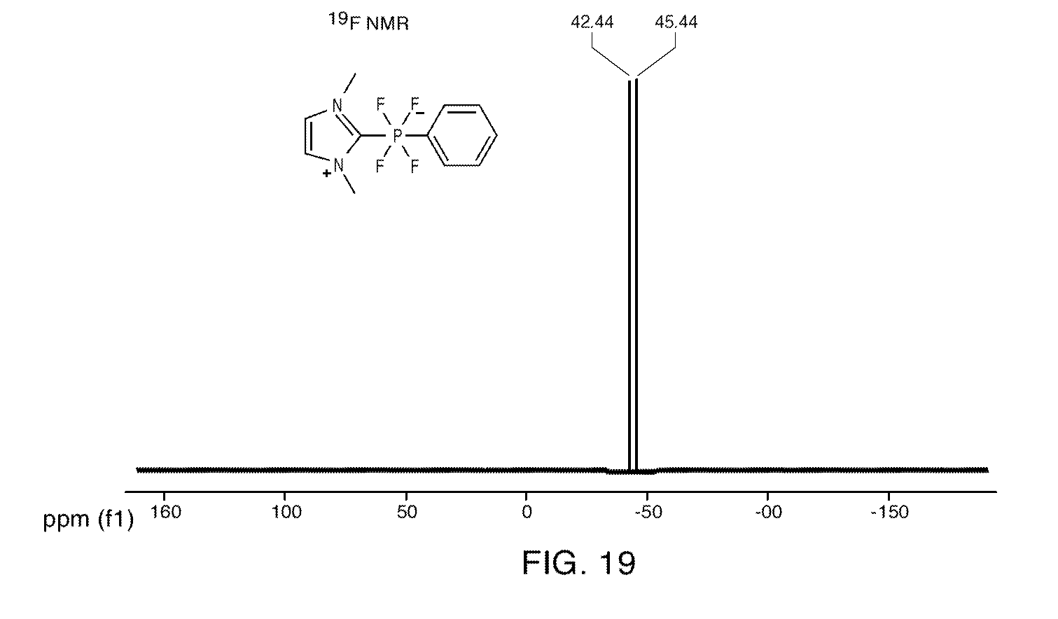

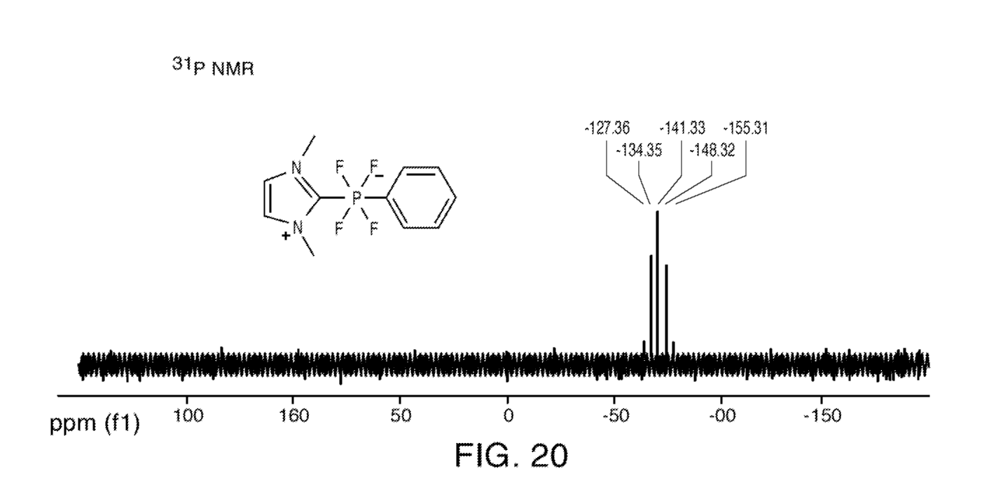

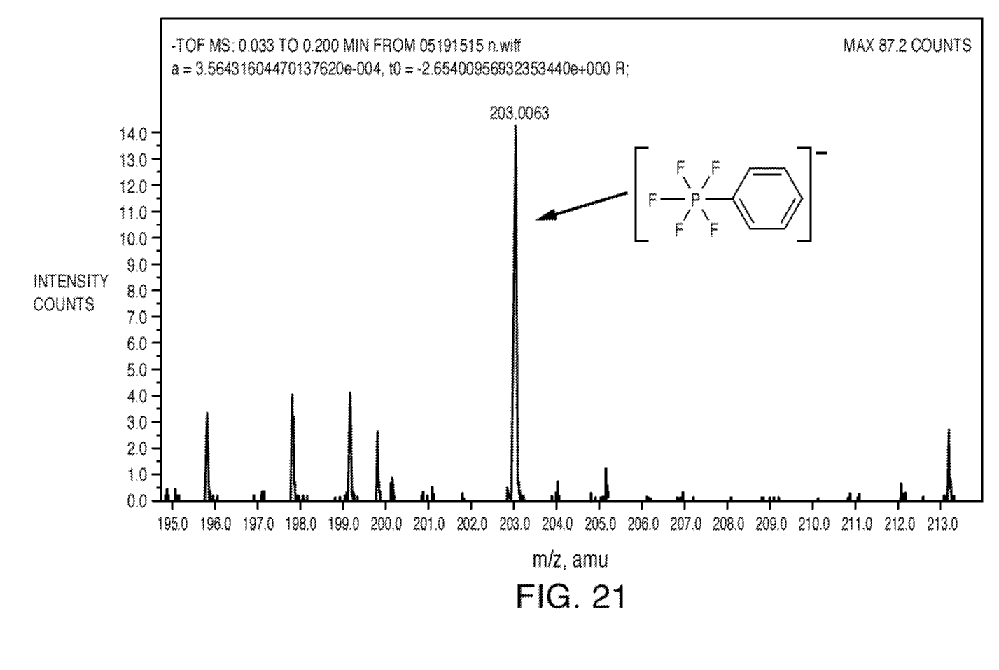

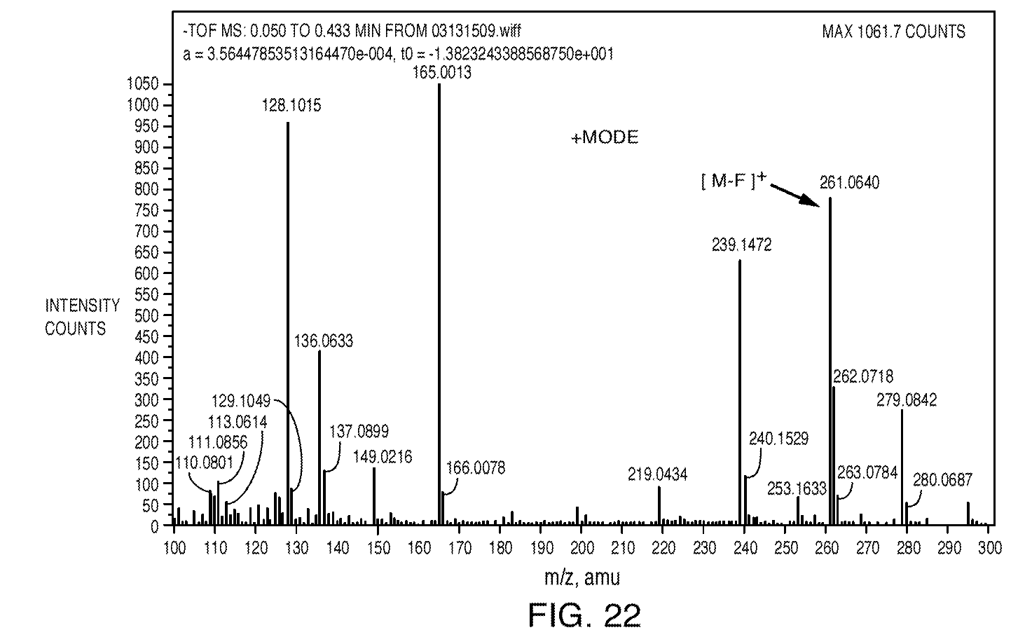

The hydrolytic stability study of 1 and 2 was evaluated using a previously published method [50]. The compounds were dissolved in D₂O-CD₃CN (8/2 vol) at pH 7.5 ([phosphate buffer]=500 mM) and the hydrolysis reaction was monitored by ¹⁹F NMR spectroscopy. While salt K[PF₅Ph] shows a complete hydrolysis in less than 5 min, both carbenes adducts 1 and 2 are highly water stable. Compound 2 undergoes a slow hydrolysis to afford free fluoride and phosphate with a pseudo-first order rate constant (k_{obs}) of 2.3×10⁻⁵ min⁻¹ (FIG. 8, Table 1). Surprisingly, free fluoride signal for 1 was not observed after five days indicating that 1 can be considered as “eternal” (FIG. 9). It is more stable than the BF₃ analogue which shows a hydrolytic rate constant (k_{Obs}) of 1.2×10⁻⁶ min⁻¹ under the same conditions [41].

TABLE 1
Hydrolytic kinetics of 2. The values provided for int [F⁻] and int [2]
correspond to the integration of the corresponding ¹⁹F NMR signal.

| Time | | | [2]/([2] + [F⁻]) | [2]/([2] + [F⁻]) | |
|---|---|---|---|---|---|
| (min) | int[F⁻] | int[2] | exp | calc | ln[2] |
| 0 | 0 | 100 | 1.000 | 1.000 | 0.000 |
| 5 | 2 | 100 | 0.979 | 1.000 | −0.021 |
| 1035 | 5 | 100 | 0.954 | 0.976 | −0.048 |
| 2940 | 10 | 100 | 0.912 | 0.933 | −0.092 |
| 8380 | 22 | 100 | 0.816 | 0.821 | −0.203 |
| 12715 | 37 | 100 | 0.729 | 0.742 | −0.317 |
| 20050 | 63 | 100 | 0.612 | 0.624 | −0.491 |
| 28759 | 99 | 100 | 0.502 | 0.509 | −0.690 |

As illustrated in Table 2, the radiochemical yields (RCY) of 1 which was calculated based on the radio-activity of the isolated product and the starting radio-activity are quite low (4-6% decay corrected RCY). These low yields originate from the stability of the P—F bonds which impedes the ¹⁸F-¹⁹F isotopic exchange process. It was found that increasing the reaction temperature leads to higher radiochemical yield (Table 2, entries 1-3). However, when a high reaction temperature (100° C.) was employed, the radio-peak of [¹⁸F]-1 and the UV-peak of I were not observed by HPLC suggesting precursor decomposition (FIG. 10 A-B). Similar issues were encountered in the radiofluorination of 2, for which all efforts proved unsuccessful including those involving different types of activators such as SnCl₂, SnCl₄, TMSOTf, HCl, and KHF₂.

TABLE 2
Radiosynthetic results for [¹⁸F]- 1

| | [1] | SnCl₄ | Temp. | Time | SA^{a} | RCY^{b} |
|---|---|---|---|---|---|---|
| Entry | (μmol) | (equiv.) | (° C.) | (Min) | (mCi/μmol) | (%) |
| 1 | 0.9 | 5 | 25 | 10 | No [¹⁸F]-1 observed | |
| 2 | 0.9 | 5 | 60 | 10 | 22.7 | 4.3 |
| 3 | 0.9 | 5 | 80 | 10 | 32.5 | 6.6 |

^{a}Specific activity is determined by dividing the product activity by the amount of the product (based on the integration of UV-HPLC and compare with the UV chromatogram of the standard).
^{b}RCY = activity of the isolated product/starting ¹⁸F activity. All yields are decay corrected.

TABLE 3
Radiosynthetic results for [¹⁸F]-1

| | | | | | | | Post Sep-Pak | | | |
|---|---|---|---|---|---|---|---|---|---|---|
| Pre Sep-Pak purification | | | | | | | purification | | Post HPLC | |
| | Starting | Amount | MeCN | | | | | solution | Amount | |
| | activity | of [1] | Volume | SnCl₄ | Temp | Time | Activity | volume | of [1] | Activity |
| Entry | (mCi) | (μmol) | (μL) | (eq) | (° C.) | (min) | (mCi) | (mL) | (μmol) | (mCi) |
| 1 | 98.8 | 0.9 | 30 | 5 | 25 | 10 | No [¹⁸F]- 1 observed | | | |
| 2 | 390 | 0.9 | 30 | 5 | 60 | 10 | 18 | 1 | 0.74 | 16.8 |
| 3 | 370 | 0.9 | 30 | 5 | 80 | 10 | 27.7 | 1 | 0.75 | 24.4 |

| | | | | | | | |
|---|---|---|---|---|---|---|---|
| 4 | 102.5 | 0.9 | 30 | 5 | 100 | 30 | No [¹⁸F]- 1 observed |

The stability of [¹⁸F]-1 was first investigated in phosphate buffer solution (1×PBS). [¹⁸F]-1 displayed >98% radiochemical purity even after an incubation time of 3 hours. This result suggested that [¹⁸F]-1 might be extremely stable under physiological condition. The stability of [¹⁸F]-1 was further evaluated in a murine model. The probe [¹⁸F]-1 (0.1 mCi) was injected into female nude mice and static microPET scans were obtained at 3 hours after the injection. As shown in FIG. 11 A-B, the microPET/CT images showed an obvious localization in the bladder indicating that [¹⁸F]-1 was cleared through the urinary track. No bone uptake was observed suggesting that the [¹⁸F]-fluoride release was insignificant even 3 hours post-injection.

In conclusion, a phosphorus-based [¹⁸F]-radiotracer was synthesized. Owing to Coulombic effects between the imidazolium and phosphate moieties, this probe is remarkably resistant to hydrolysis. Although it is not necessary to understand the mechanism of an invention, it is believed that such probes can be radiolabeled by isotopic exchange when SnCl₄ is used as an acidic promoter and can be imaged using PET for as long as three hours post injection. It is further believed that it is possible that there are additional ways to functionalize this adduct such that it can be used as a prosthetic group for targeted tissue and disease imaging.

### EXAMPLES

The following examples are provided in order to demonstrate and further illustrate certain preferred embodiments and aspects of the present invention and are not to be construed as limiting the scope thereof.

### Example 1

### Experimental: General Procedures

1-Methylimidazole and methyl iodide, from Alfar Aesar, Sodium acetate was purchased from Mallinckrodt. Dichlorophenylphosphine, and bromine was purchased from Strem Chemicals. Potassium fluoride was purchased from Sigma Aldrich. All chemicals were used without further purification. Potassium fluoride was stored in an oven at 100° C. and dried under vacuum at 100° C. for 2 h before use. Solvents were dried by passing through an alumina column (CH₂Cl₂), refluxing under N₂ over Na (Et₂O and THF), refluxing under N₂ over CaH₂ and stored on 3 Å molecular sieves (CH₃CN). Electrospray mass spectra were acquired on a MDS Sciex API QStar Pulsar. NMR spectra were recorded on a Varian Unity Inova 300 NMR and an Inova 5008 spectrometer at ambient temperature. Chemical shifts are given in ppm, and are referenced to residual ¹H and ¹³C solvent signals as well as external BF₃-Et₂O (¹⁹F NMR) and H₃PO₄ (³¹P NMR).

Previously published procedures were followed for compound 1 [51] and dimethylimidazolium iodide [52].

### Example 2

### Procedure for KPF₅Ph and (NHC)PF₄Ph (2) Synthesis

KPF₅Ph.

Bromine (6.2 mL, 120 mmol) was added to a mixture of potassium fluoride (42 g, 723 mmol) and dichlorophenylphosphine (16.3 mL, 120 mmol) in acetonitrile (250 mL), which caused instantaneously a color change to yellow. The mixture was stirred at room temperature for 18 h to give a dark brown a solution with a white precipitate. Volatiles were evaporated under vacuum, extract with acetonitrile (2×100 mL), and filtered. Evaporation of the solvent was followed by washing of the solid residue with EtO (2×50 mL), and drying under vacuum gave the desired product as a white powder (26.7 g, 92%). X-ray quality crystals were obtained from a saturated solution in acetonitrile at −18° C. This compound must be protected from ambient atmosphere, because it appeared to be hydrolysed: the white powder may become an acidic oil (pH<2) after 15 min exposure to air. ¹H NMR (500 MHz, CD₃CN): δ7.23-7.30 (m, 3H, H^{ortho+para}), 7.63-7.68 (m, 2H, H^{meta}). ³¹P NMR (202 MHz, CD₃CN): δ−137.0 (quintd, J_{PF}=673 Hz, J_{PF}=822 Hz). ¹⁹F NMR (470 MHz, CD₃CN): δ−58.4 (dd, 4F, J_{FF}=822 Hz, J_{FF}36 Hz), −61.1 (dquint, 1F, J_{FP}=673 Hz, J_{FF}=36 Hz), see FIG. 13. ¹³C NMR (125 MHz, CD₃CN): δ 127.79 (d, J_{CF}=19 Hz, CH^{ortho}), 127.90 (m, CH^{meta}), 131.47 (dquint, J_{CF}=4.2 Hz, J_{CF}=9.3 Hz, CH^{para}), 150.31 (dquint, J_{CF}=306 Hz, J_{CF}=45 Hz, C^{ipso}), HRMS (ESI−) calcd for [M]⁺: 203.0063, found: 203.0049. Anal. Calcd. for C₆H₅F₅KP (242.17): C, 29.76; H, 2.08. Found: C, 29.91; H, 1.98.

### Example 3

### Synthesis of (NHC)PF₄Ph, (2)

A 2.2 M solution of n-BuLi in hexane (4.54 mL, 10 mmol) was added dropwise at −78° C. to a heterogeneous mixture of KPF₅Ph (2.42 g, 10 mmol) and dimethylimidazolium iodide (2.24 g, 10 mmol) in THF (50 mL). The solution was slowly reheated at room temperature then heated for 18 h at 65° C. The volatiles were evaporated under vacuum, the solid residue was washed with several portions of water (100 mL), filtered, washed with a small portion of EtOH (10 mL), and dried under vacuum to give a white powder (1.83 g, 65%). X-ray quality crystals were obtained by slow evaporation of a solution of acetonitrile under ambient atmosphere. ¹H NMR (300 MHz, CD₃CN): δ 3.97 (s, 6H, CH₃), 7.09 (d, J_{PH}=3.1 Hz, 2H, CH^{NHC}), 7.24-7.31 (m, 3H, H^{Ph-ortho+para}), 7.65-7.70 (m, 2H, H^{Ph-meta}). ³¹P NMR (121 MHz, CD₃CN): δ−141.1 (quint, J_{PF}=849 Hz). ¹⁹F {¹H} NMR (282 MHz, CD₃CN): δ −43.9 (d, J_{FP}=849 Hz). ¹³C {¹H} NMR (75 MHz, CD₃CN): δ 39.10 (quint, J_{CF}=4.4 Hz, CH₃), 123.09 (d, J_{CP}=9.9 Hz, CH^{NHC}), 127.93 (d, J_{CP}=20.3 Hz, CH^{Ph-para}), 128.26 (d, J_{CF}=4.0 Hz, CH^{Ph-meta}), 131.49 (dquint, J_{CF}=4.0 Hz, J_{CP}=11.3 Hz, CH^{Ph-para}), 150.01 (dquint, J_{CF}=43 Hz, J_{CP}=297 Hz, CH^{Ph-iso}), 159.84 (dquint, J_{CF}=71 Hz, J_{CF}=334 Hz, C_{q}^{NHC}). HRMS (ESI+) calcd for [M−F]⁺: 261.0768, found: 261.0640. Anal. Calcd. for C₁₁H₁₃F₄N₂P (280.21): C, 47.15; H, 4.68. Found: C, 47.05; H, 4.57.

### Example 4

### Crystal Structure Determinations.

The crystallographic measurement of KPF₅Ph (FIG. 5) and 2 (FIG. 4) were performed using a Bruker APEX-II CCD area detector diffractometer, with graphite-monochromated Mo-Kα radiation (λ=0.71069 Å). A specimen of suitable size and quality was selected and mounted onto a nylon loop. The semi-empirical method SADABS was applied for absorption correction. The structure was solved by direct methods, and refined by the full-matrix least-square method against F2 with the anisotropic temperature parameters for all non-hydrogen atoms. All H atoms were geometrically placed and refined using the riding model approximations. Data reduction and further calculations were performed using the Bruker SAINT+ and SHELXTL NT program packages. Table 4 includes crystal data collection and refinement parameters for compounds 2 and KPF₅Ph. Table 5 includes selected distances (Å) and angles (°) for KPF₅Ph.

TABLE 4
Crystal data collection and refinement parameters for compounds
2 and KPF₅Ph.

| | 2 | | KPF₅Ph | |
|---|---|---|---|---|
| chemical formula | C₁₁H₁₃F₄N₂P | | C₃₂H₃₂F₂₀K₄N₄P₄ | |
| Fw | 280.2 | | 1132.9 | |
| T (K) | 110 | (2) | 110 | (2) |
| wavelength (Å) | 0.71073 | | 0.71073 | |
| space group | P21/n | | P21/n | |
| a (Å) | 7.5298 | (13) | 21.180 | (3) |
| b (Å) | 11.1769 | (19) | 8.8602 | (13) |
| c (Å) | 14.477 | (3) | 24.220 | (4) |
| α (deg) | 90 | | 90 | |
| β (deg) | 104.313 | (2) | 102.725 | (2) |
| γ (deg) | 90 | | 90 | |
| Z | 4 | | 4 | |
| V (Å³) | 1180.6 | (4) | 4433.4 | (11) |

| | | |
|---|---|---|
| ρ_{calcd} (g cm⁻³) | 1.577 | 1.697 |
| μ (mm⁻¹) | 0.268 | 0.662 |
| θ range (deg) | 2.33-28.29 | 1.97-27.25 |
| R1^{a} [I > 2σ(I)] | 0.0352 | 0.0418 |
| wR2^{b} [I > 2σ(I)] | 0.0954 | 0.0952 |
| R1 [all data] | 0.0425 | 0.0594 |
| wR2 [all data] | 0.1004 | 0.1036 |
| GOF | 1.069 | 1.036 |

^{a}R₁ = Σ(||Fₒ| − |F_{c}||)/Σ|Fₒ|
^{B}WR₂ = {Σ[WFo² − Fc²)²]/Σ[WFo²)²]}^{1/2}

TABLE 5
Selected distances (Å) and angles (°) for KPF₅Ph.

| | Molecule 1 | Molecule 2 | Molecule 3 | Molecule 4 |
|---|---|---|---|---|
| P—C | P₁—C₁ = 1.837(3) | P₂—C₇ = 1.829(3) | P₃—C₁₃ = 1.831(3) | P₄—C₂₇ = 1.833(3) |
| P—Fₜᵣₐₙₛ | P₁—F₁ = 1.6391(16) | P₂—F₆ = 1.5857(19) | P₃—F₁₁ = 1.6512(17) | P₄—F₁₆ = 1.6323(16) |
| P—F_{cis} | P₁—F₂ = 1.6187(17) | P₂—F₇ = 1.6460(15) | P₃—F₁₂ = 1.6064(17) | P₄—F₁₇ = 1.6394(15) |
| | P₁—F₃ = 1.6121(16) | P₂—F₈ = 1.6287(16) | P₃—F₁₃ = 1.6132(16) | P₄—F₁₈ = 1.6203(15) |
| | P₁—F₄ = 1.6288(17) | P₂—F₉ = 1.6318(16) | P₃—F₁₄ = 1.6292(16) | P₄—F₁₉ = 1.6281(15) |
| | P₁—F₅ = 1.6263(16) | P₂—F₁₀ = 1.6367(17) | P₃—F₁₅ = 1.6236(17) | P₄—F₂₀ = 1.6321(15) |
| C—P—Fₜᵣₐₙₛ | C₁—P₁—F₁ = | C₇—P₂—F₆ = | C₁₃—P₃—F₁₁ = | C₂₇—P₄—F₁₆ = |
| | 178.84(11) | 178.68(12) | 179.15(12) | 179.70(11) |
| C—P—F_{cis} | C₁—P₁—F₂ = | C₇—P₂—F₇ = | C₁₃—P₃—F₁₂ = | C₂₇—P₄—F₁₇ = |
| | 92.63(10) | 92.60(10) | 93.57(11) | 92.45(10) |
| | C₁—P₁—F₃ = | C₇—P₂—F₈ = | C₁₃—P₃—F₁₃ = | C₂₇—P₄—F₁₈ = |
| | 93.02(10) | 92.86(10) | 93.50(11) | 92.92(10) |
| | C₁—P₁—F₄ = | C₇—P₂—F₉ = | C₁₃—P₃—F₁₄ = | C₂₇—P₄—F₁₉ = |
| | 91.83(10) | 92.32(10) | 92.11(10) | 92.78(10) |
| | C₁—P₁—F₅ = | C₇—P₂—F₁₀ = | C₁₃—P₃—F₁₅ = | C₂₇—P₄—F₂₀ = |
| | 93.10(10) | 92.72(10) | 92.80(10) | 93.03(10) |

Complete details of the X-ray analyses reported herein have been deposited at The Cambridge Crystallographic Data Centre (CCDC 1504580 (KPF₅Ph), 1504579 (2)). This data can be obtained free of charge via world wide web ccdc.cam.ac.uk/data_request/cif.

### Example 5

### Kinetic Studies of the Hydrolysis Reactions for 1 and 2

A sample of I was dissolved in a mixture of 0.2 mL CD₃CN and 0.8 mL D₂O phosphate buffer solution (pH 7.5, 500 mM) while a sample of 2 (5 mg), was dissolved in a mixture of 0.3 mL dmso-d6, 0.63 mL H₂O phosphate buffer (pH 7.5, 500 mM) and 70 mg of Triton X-100. The ¹⁹F NMR spectra of I and 2 were collected periodically. The decomposition of 2 were monitored by integration of the decreasing of the signal of 2 in conjunction with the increasing signal corresponding to free F′. The rate constant, k_{obs}, was calculated using a well-established NMR method reported in the literature[50]. This method was based on the fact that that the concentration of 2 is proportional to the ¹⁹F NMR integration of the signal of 2 divided by the sum of the integration of the signal of 2 and the free fluoride signal. For convenience, the value of the integration of 2 was arbitrarily set at 100 and the free fluoride integration determined. The resulting data is provided in Table 1.

### Example 6

### Radiochemistry Experiment

All chemicals were purchased in analytical grade and used without further purification. Analytical reversed-phase high-performance liquid chromatography (HPLC) was performed on a SPD-M30A photodiode array detector (Shimadzu) and model 105S single-channel radiation detector (Carroll & Ramsey Associates) using a Gemini 5μ C18 column (250×4.6 mm). The flow was set to 1 mL/min. The mobile phase was programmed to change from 95% solvent A and 5% solvent B (0-2 min) to 5% solvent A and 95% solvent B at 22 min, where solvent A is 0.1% TFA in water and solvent B is 0.1% TFA in acetronitrile. See FIGS. 12A&B.

### Example 7

### Radiolabeling

The radiolabeling reactions were performed using the following protocol. Compound 1 (0.9 μmol) was mixed with SnCl₄ (5 equiv.) in 30 μL of anhydrous MeCN. The resulting solution was then combined with [¹⁸F]-tetra-n-butylammonium fluoride (TBAF) in MeCN. After incubating at reaction temperature (room temperature, 60° C., 80° C., or 100° C.) for 10 min, the reaction was quenched by adding 10 mL of water. The mixture was passed through a Sep-Pak cartridge (Sep-Pak Plus tC18) and washed with another 10 mL of water to remove all Sn-by-products. The radiolabeled derivative [¹⁸F]-1 was eluted off the cartridge by 1 mL of MeCN.

### Example 8

### In Vitro Stability Test

After HPLC purification, [¹⁸F]-1 was re-injected into HPLC for a radio profile standard. Then, the probe was added with 10×PBS to reconstruct the solution to IX PBS and 0.1 N NaOH to adjust pH to 7, respectively. After 1 hour and 3 hours incubation, a fraction of [¹⁸F]-1 was injected into HPLC. The radio purity was calculated based on the integration of the product peak and other minor peaks.

### Example 9

### MicroPET Imaging

MicroPET imaging were acquired at 3 h post injection. For PET image acquiring, a female nude mouse was injected with 0.1 mCi of [¹⁸F]-1 via the tail vein. At 3 hour post injection, the mouse was anesthetized using isoflurane (2% in oxygen), then placed into imaging chambers equipped with a heated coil to maintain body temperature and gas anesthesia. The static microPET acquisitions were then achieved and reconstructed for analysis.

Thus, specific compositions and methods of probes for ^{18F} positron emission tomography imaging have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

1. Merrifield, R. B. (1963) “Solid Phase Peptide Synthesis. I. The Synthesis of a Tetrapeptide,” J. Am. Chem. Soc. 85(14), 2149-2154.
2. Battle, M. R. et al. (2011) “Monitoring Tumor Response to Antiangiogenic Sunitinib Therapy with 18F-Fluciclatide, an 18F-Labeled Avp3-lntegrin and Avl5-Integrin Imaging Agent,” J. Nucl. Med. 52(3), 424-430.
3. Morrison, M. and Cuthbertson, A. (2011) “Integrin Imaging to Evaluate Treatment Response,” Theranostics 1, 149-153.
4. Herrmann, W. A. and Köcher, C. (1997) “N-Heterocyclic Carbenes,” Angew Chem. Int. Ed. 36(20), 2162-2187.
5. Hopkinson, M. N. et al. (2014) “An Overview of N-Heterocyclic Carbenes,” Nature 510(7506), 485-496.
6. Satyamurthy, N. et al. (1999) “Electronic Generators for the Production of Positron-Emitter Labeled Radiopharmaceuticals: Where Would PET Be without Them?,” Clinical Positron Imaging 2(5), 233-253.
7. Wuts, P. G. M. and Greene, T. W. (2006) Greene's Protective Groups in Organic Synthesis, 4th ed., John Wiley & Sons, Inc., Hoboken, N.J.
8. McBride, W. J. et al. “Al—F-18-Labeled, Al—F-19-Labeled and Ga-68-Labeled Gastrin-Releasing Peptide Receptor (Grpr)-Antagonists for Imaging of Prostate Cancer,” United States Patent Application Publication Number US 2015-0217006 A1, application Ser. No. 14/599,816, filed Jan. 19, 2015. (published Aug. 6, 2015).
9. D'Souza, C. A. et al. “Methods and Compositions for Improved F-18 Labeling of Proteins, Peptides and Other Molecules,” United States Patent Application Publication Number US 2013-0315821 A1, application Ser. No. 13/897,849, filed 5120:2013. (published Nov. 28, 2013).
10. D'Souza, C. A. et al. “Methods and Compositions for Improved F-18 Labeling of Proteins, Peptides and Other Molecules,” WIPO PCT Patent Publication Number WO/2012/082618, Application PCT/US2011/064407, filed Dec. 12, 2011. (published 6/212012).
11. D'Souza, C. A. et al. “Methods and Compositions for Improved F-18 Labeling of Proteins, Peptides and Other Molecules,” United States Patent Application Publication Number US 2012-0134920 A1, application Ser. No. 13/323,139, filed Dec. 12, 2011. (published May 31, 2012).
12. McBride, W. J. et al. “Methods and Compositions for F-18 Labeling of Proteins, Peptides and Other Molecules,” United States Patent Application Publication Number US 2009-0246130 A1, application Ser. No. 12/433,212, filed Apr. 30, 2009. Oct. 1, 2009).
13. McBride, W. J. and Goldenberg, D. M. “Improved Methods and Compositions for F-18 Labeling of Proteins, Peptides and Other Molecules,” WIPO PCT Patent Publication Number WO2009/079024, Application PCT/US2008/062108, filed 4/3012008. (published Jun. 25, 2009).
14. McBride, W. J. et al. “Methods and Compositions for F-18 Labeling of Proteins, Peptides and Other Molecules,” United States Patent Application Publication Number US 2009-0155166 A1, application Ser. No. 12/343,655, filed Dec. 24, 2008. Jun. 18, 2009).
15. McBride, W. J. and Goldenberg, D. M. “Methods and Compositions for F-18 Labeling of Proteins, Peptides and Other Molecules,” United States Patent Application Publication Number US 2008-0253964 A1, application Ser. No. 12/112,289, filed Apr. 30, 2008. (published Oct. 16, 2008).
16. Perrin, D. et al. “Substituted Aryl-Fluoroborates as Imaging Agents,” U.S. Pat. No. 8,574,546, application Ser. No. 12/670,108, filed Jun. 22, 2010. (issued Nov. 5, 2013).
17. Perrin, D. M. and Ting, R. “Radiolabeled Compounds and Compositions, Their Precursors and Methods for Their Production,” U.S. Pat. No. 8,114,381, application Ser. No. 10/589,220, filed May 21, 2007. (issued Feb. 14, 2012).
18. Perrin, D. and Liu, Z. “Substituted Organofluoroborates as Imaging Agents,” WIPO PCT Patent Publication Number WO2014/134716, Application PCT/CA20141000200, filed Mar. 7, 2014. (published Sep. 12, 2014).
19. Perrin, D. et al. “Substituted Aryl-Fluoroborates as Imaging Agents,” WIPO PCT Patent Publication Number WO/2009/012596, Application PCT/CA2008/001368, filed Jul. 24, 2008. (published Jan. 29, 2009).
20. Perrin, D. M. and Ting, R. “Radiolabeled Compounds and Compositions, Their Precursors and Methods for Their Production,” WIPO PCT Patent Publication Number WO/2005/077967, Application PCT/CA2005/000195, filed Feb. 14, 2005. (published Aug. 25, 2005).
21. Miller, P. W. et al. (2008) “Synthesis of C-11, F-18, O-15, and N-13 Radiolabels for Positron Emission Tomography,” Angew. Chem. Int. Ed. 47(47), 8998-9033.
22. Chansaenpak, K. et al. (2016) “[¹⁸F]-Group 13 Fluoride Derivatives as Radiotracers for Positron Emission Tomography,” Chem. Soc. Rev. 45(4), 954-971.
23. Bernard-Gauthier, V. et al. (2016) “From Unorthodox to Established: The Current Status of 18F-Trifluoroborate- and 18F-Sifa-Based Radiopharmaceuticals in PET Nuclear Imaging,” Bioconjug. Chem. 27(2), 267-279.
24. Perrin, D. M. (2016) “[18F]-Organotrifluoroborates as Radioprosthetic Groups for PET Imaging: From Design Principles to Preclinical Applications,” Acc. Chem. Res.
25. Liu, Z. et al. (2014) “A New F-18-Heteroaryltrifluoroborate Radio-Prosthetic with Greatly Enhanced Stability That Is Labelled by F-18-F-19-Isotope Exchange in Good Yield at High Specific Activity,” Medchemcomm 5(2), 171-179.
26. Liu, Z. B. et al. (2013) “Stoichiometric Leverage: Rapid 18F-Aryltrifluoroborate Radiosynthesis at High Specific Activity for Click Conjugation,” Angew Chem. Int. Ed. 52(8), 2303-2307.
27. Li, Y. et al. (2011) “Towards Kit-Like F-18-Labeling of Marimastat, a Noncovalent Inhibitor Drug for in vivo PET Imaging Cancer Associated Matrix Metalloproteases,” Medchemcomm 2(10), 942-949.
28. Keller, U. A. D. et al. (2010) “Novel Matrix Metalloproteinase Inhibitor [F-18]Marimastat-Aryltrifluoroborate as a Probe for in vivo Positron Emission Tomography Imaging in Cancer,” Cancer Res. 70(19), 7562-7569.
29. Ting, R. et al. (2008) “Capturing Aqueous [(18)F]-Fluoride with an Arylboronic Ester for PET: Synthesis and Aqueous Stability of a Fluorescent [(18)F]-Labeled Aryltrifluoroborate,” J. Fluorine Chem. 129(5), 349-358.
30. Ting, R. et al. (2008) “Toward [(18)F]-Labeled Aryltrifluoroborate Radiotracers: In vivo Positron Emission Tomography Imaging of Stable Aryltrifluoroborate Clearance in Mice,” J. Am. Chem. Soc. 130(36), 12045-12055.
31. Ting, R. et al. (2005) “Arylfluoroborates and Alkylfluorosilicates as Potential PET Imaging Agents: High-Yielding Aqueous Biomolecular F-18-Labeling,” J. Am. Chem. Soc. 127(38), 13094-13095.
32. Li, Z. B. et al. (2011) “Rapid Aqueous [F-18]-Labeling of a Bodipy Dye for Positron Emission Tomography/Fluorescence Dual Modality Imaging,” Chem. Commun. 47(33), 9324-9326.
33. Schirrmacher, E. et al. (2007) “Synthesis of P-(Di-Tert-Butyl[(18)F]Fluorosilyl)Benzaldehyde ([F-18]Sifa-a) with High Specific Activity by Isotopic Exchange: A Convenient Labeling Synthon for the F-18-Labeling of N-Amino-Oxy Derivatized Peptides,” Bloconjug. Chem. 18(6), 2085-2089.
34. Schirrmacher, R. et al. (2006) “F-18-Labeling of Peptides by Means of an Organosilicon-Based Fluoride Acceptor,” Angew. Chem. Int. Ed. 45(36), 6047-6050.
35. Liu, Z. et al. (2014) “Preclinical Evaluation of a High-Affinity 18F-Trifluoroborate Octreotate Derivative for Somatostatin Receptor Imaging,” J. Nucl. Med. 55(9), 1499-1505.
36. Liu, Z. B. et al. (2014) “An Organotrifluoroborate for Broadly Applicable One-Step F-18-Labeling,” Angew. Chem. Int. Ed. 53(44), 11876-11880.
37. Pourghiasian, M. et al. (2015) “18F-Ambf3-Mj9: A Novel Radiofluorinated Bombesin Derivative for Prostate Cancer Imaging,” Bioorg. Med. Chem. 23(7), 1500-1506.
38. Liu, Z. et al. (2015) “18F-Trifluoroborate Derivatives of [Des-Argl)O]Kallidin for Imaging Bradykinin B Receptor Expression with Positron Emission Tomography,” Mol. Pharm. 12(3), 974-982.
39. Liu, Z. et al. (2015) “One-Step 18F Labeling of Biomolecules Using Organotrifluoroborates,” Nat. Protoc. 10(9), 1423-1432.
40. Chansaenpak, K. et al. (2016) “Synthesis and in vivo Stability Studies of [18F]-Zwitterionic Phosphonium Aryltrifluoroboratellndomethacin Conjugates,” RSC Advances 6(28), 23126-23133.
41. Chansaenpak, K. et al. (2015) “[18F]-Nhc-Bf3 Adducts as Water Stable Radio-Prosthetic Groups for PET Imaging,” Chem. Commun. 51(62), 12439-12442.
42. Li, Z. B. et al. (2012) “Harvesting F-18-Fluoride Ions in Water Via Direct F-18-F-19 Isotopic Exchange: Radiofluorination ofZwitterionic Aryltrifluoroborates and in vivo Stability Studies,” Medchemcomm 3(10), 1305-1308.
43. Hudnall, T. W. et al. (2008) “Fluoride Ion Chelation by a Bidentate Phosphonium/Borane Lewis Acid,” J. Am. Chem. Soc. 130(33), 10890-10891.
44. Caputo, C. B. et al. (2013) “Lewis Acidity of Organofluorophosphonium Salts: Hydrodefluorination by a Saturated Acceptor,” Science 341, 1374-1377.
45. Bayne, J. M. and Stephan, D. W. (2016) “Phosphorus Lewis Acids: Emerging Reactivity and Applications in Catalysis,” Chem. Soc. Rev 45(4), 765-774.
46. Studenov, A. R. et al. (2005) “New Radiolabelling Chemistry: Synthesis of Phosphorus-[18F]Fluorine Compounds,” J. Labelled Compd. Radiopharm. 48(7), 497-500.
47. Ghorab, M. F. and Winfield, J. M. (1990) “Radiotracers in Fluorine Chemistry. Part Xlv. The Lability of the Fluoroanions Bf₄⁻, Pf₆⁻, Asf₆⁻, Sbf₆, Nbf₆⁻ and Taf₆⁻ in Acetonitrile Solution. A Fluorine-18 Radiotracer Study [1,2],” J. Fluorine Chem. 49(3), 367-383.
48. Schmutzler, R. (1964) “Phenylbis(Dimethylamino)Fluorophosphonium Phenylpentafluorophosphate,” J. Am. Chem. Soc. 86(20), 4500-4502.
49. Böttcher, T. et al. (2012) “Carbene Complexes of Phosphorus(V) Fluorides by Oxidative Addition of 2,2-Difluorobis(Dialkylamines) to Phosphorus(III) Halides,” Organometallics 31(4), 1278-1280.
50. Ting, R. et al. (2008) “Substituent Effects on Aryltrifluoroborate Solvolysis in Water: Implications for Suzuki-Miyaura Coupling and the Design of Stable F-18-Labeled Aryltrifluoroborates for Use in PET Imaging,” J. Org. Chem. 73(12), 4662-4670.
51. Tian, C. et al. (2012) “High-Yield Thermolytic Conversion of Imidazolium Salts into Arduengo Carbene Adducts with Bf3 and Pf5,” Organometallics 31(5), 1751-1760.
52. Zoller, U. (1988) “The Cheletrofic Fragmentation of Hypervalent Three-Membered Thiahetesocyclic Intermediates,” Tetrahedron 44(24), 7413-7426.

We claim:

1. A compound with the structure:

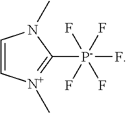

2. The compound of claim 1, wherein at least one F is ¹⁸F

3. A compound with the structure:

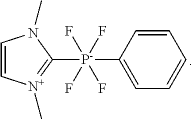 [SMILES: Cn1cc[n+](C)c1[P-](F)(F)(F)(F)c1ccccc1]

4. The compound of claim 3, wherein at least one F is ¹⁸F

5. A method for the radiofluorination of a phosphorus(V) fluoride compound comprising:
(a) providing:
(i) a phosphorus(V) fluoride compound, and
(ii) an [¹⁸F] source,
(iii) an isotopic exchange promoter, and
(b) isotopically exchanging the fluoride in said phosphorus(V) fluoride compound with the [¹⁸F] of said [¹⁸F] source with said isotopic exchange promoter to create an [¹⁸F]phosphorus(V) fluoride compound.

6. The method of claim 5, wherein said phosphorus(V) fluoride compound comprises a zwitterionic compound containing a formally anionic phosphorus(V) fluoride moiety bound to a formally cationic group

7. The method of claim 6, wherein said cationic group is selected from the group consisting of an ammonium, an iminium, an anilinium, a phopshoniun, a sulfonium, an arsonium, a stibonium, a selenonium, and a telluronium.

8. The method of claim 6, wherein said cationic group comprises organic groups amenable to facile conjugation with biomolecules.

9. The method of claim 8, wherein said organic groups amenable to facile conjugation with biomolecules are terminated by a functional group selected from the consisting of an alkyne, an azide, a thiol, a caroxilic acid, an N-succinimde ester, a maleimide, a sulfonate, a triflate, and an amine.

10. The method of claim 5, wherein said phosphorus(V) fluoride compound is a N-heterocyclic carbene phosphorus(V) fluoride derivative.

11. The method of claim 5, wherein said isotopic exchange promoter is SnCl₄.

12. The method of claim 11, wherein said method further comprises step (c) quenching with water.

13. The method of claim 5, wherein said [¹⁸F] source is [¹⁸F]-tetra-n-butylammonium fluoride.

14. The method of claim 10, wherein said N-heterocyclic carbene phosphorus(V) fluoride derivative is

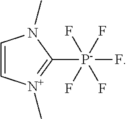

15. The method of claim 10, wherein said N-heterocyclic carbene phosphorus(V) fluoride derivative is

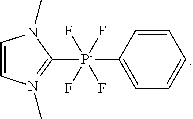 [SMILES: Cn1cc[n+](C)c1[P-](F)(F)(F)(F)c1ccccc1]

16. A method for preparing ¹⁸F-phosphorous-based radiotracers comprising:
(a) providing;
(i) a phosphorus(V) fluoride compound;
(ii) an [¹⁸F] source;
(iii) an isotopic exchange promoter;
(iv) a biomolecule;
(b) isotopically exchanging the fluoride in said phosphorus(V) fluoride compound with the [¹⁸F] of said [¹⁸F] source with said isotopic exchange promoter to create an [¹⁸F]phosphorus(V) fluoride compound;
(c) quenching the exchange reaction with water,
(d) isolating said [¹⁸F] phosphorus(V) fluoride compound, and
(e) attaching said [¹⁸F] phosphorus(V) fluoride compound to said biomolecule so as to produce a ¹⁸F-phosphorous-based radiotracer.

17. The method of claim 16, wherein said phosphorus(V) fluoride compound is a N-heterocyclic carbene phosphorus(V) fluoride derivative.

18. The method of claim 16, wherein said isotopic exchange promoter is SnCl₄.

19. The method of claim 16, wherein said source of [¹⁸F] is [¹⁸F]-tetra-n-butylammonium fluoride.

20. The method of claim 16, wherein said N-heterocyclic carbene phosphorus(V) fluoride derivative is

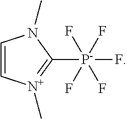

21. The method of claim 16, wherein said N-heterocyclic carbene phosphorus(V) fluoride derivative is

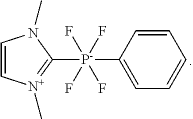 [SMILES: Cn1cc[n+](C)c1[P-](F)(F)(F)(F)c1ccccc1]

22. The method of claim 16, wherein said ¹⁸F-phosphorous-based radiotracer is for PET imaging applications

23. A method for synthesizing a compound with the structure 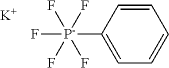 [SMILES: [K+] ; F[P-](F)(F)(F)(F)c1ccccc1]
comprising:
a) providing:
i) dichlorophenylphosphine;
ii) potassium fluoride; and
iii) bromine;
b) adding bromine to a mixture of potassium fluoride and dichlorophenylphosphine to produce a first reaction mixture;
c) stirring said first reaction mixture under such conditions that a reaction occurs;
d) evaporating volatile compounds produced by said reaction to produce a residue;
e) dissolving said residue in acetonitrile to produce a solution;
f) filtering said solution;
g) evaporating volatile compounds from said solution to produce a solid residue.
h) washing said solid residue with diethyl ether, and
i) drying said product with the structure

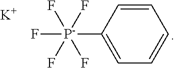 [SMILES: [K+] ; F[P-](F)(F)(F)(F)c1ccccc1]

24. A method for synthesizing a compound with the structure 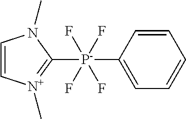 [SMILES: Cn1cc[n+](C)c1[P-](F)(F)(F)(F)c1ccccc1]
comprising:
a) providing:
i) a compound with the structure 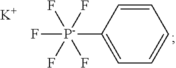 [SMILES: [K+] ; F[P-](F)(F)(F)(F)c1ccccc1]
ii) n-Butyllithium; and
iii) dimethylimidazolium iodide;
b) adding said n-Butyllithium a mixture of 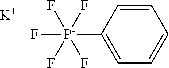 [SMILES: [K+] ; F[P-](F)(F)(F)(F)c1ccccc1]
and dimethylimidazolium iodide to produce a first reaction mixture;
c) stirring said first reaction mixture under such conditions that a reaction occurs;
d) evaporating volatile compounds produced by said reaction to produce a solid product;
e) washing said solid product with water,
f) washing said solid with ethanol, and
g) drying said product with the structure

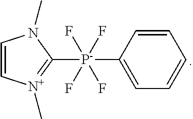 [SMILES: Cn1cc[n+](C)c1[P-](F)(F)(F)(F)c1ccccc1]

25. The method of claim 24, wherein said adding n-Butyllithium to said mixture is under −70° C.

26. The method of claim 24, wherein said conditions comprise heating said first reaction mixture to room temperature, then to 65° C.

27. A method of imaging the body of a subject comprising:
a) providing:
i) a subject comprising a tissue, and
ii) a [¹⁸F]—N-heterocyclic carbene phosphorus(V) fluoride derivative,
b) administering said [¹⁸F]—N-heterocyclic carbene phosphorus(V) fluoride derivative to said subject, and
c) imaging said tissue.

28. The method of claim 27, wherein said tissues comprises an organ.

29. The method of claim 27, wherein said [¹⁸F]—N-heterocyclic carbene phosphorus(V) fluoride derivative is in a form suitable for mammalian administration.

30. The method of claim 27, wherein said imaging comprises generating a Positron Emission Tomography image.

31. The method of claim 27, wherein said imaging is preferably carried out where the part of said tissue is diseased.

32. The method of claim 27, wherein said [¹⁸F]—N-heterocyclic carbene phosphorus(V) fluoride derivative is

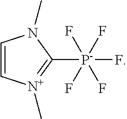

33. The method of claim 27, wherein said [¹⁸F]—N-heterocyclic carbene phosphorus(V) fluoride derivative is

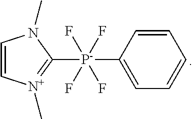 [SMILES: Cn1cc[n+](C)c1[P-](F)(F)(F)(F)c1ccccc1]

34. The method of claim 27, wherein said [¹⁸F]—N-heterocyclic carbene phosphorus(V) fluoride derivative is attached as a prosthetic group to a biomolecule.

35. A kit, comprising:
a) a first container with a N-heterocyclic carbene phosphorus(V) fluoride derivative,
b) a second container with SnCl₄,
c) a third container for a [¹⁸F] source,
d) a solid-phase extraction cartridge, and
e) instructions for use of said kit.

36. The kit of claim 35, wherein said N-heterocyclic carbene phosphorus(V) fluoride derivative is

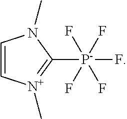

37. The kit of claim 35, wherein said N-heterocyclic carbene phosphorus(V) fluoride derivative is

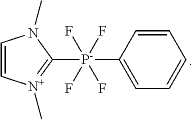 [SMILES: Cn1cc[n+](C)c1[P-](F)(F)(F)(F)c1ccccc1]

38. The kit of claim 35, wherein said kit further includes a fourth container with water.

39. The kit of claim 35, wherein said kit further includes a fifth container with anhydrous solvent.

40. The kit of claim 35, wherein said solid-phase extraction cartridge has a silica-based bonded phase with strong hydrophobicity and trifunctional bonding chemistry.

41. The kit of claim 35, wherein said instructions for use comprises:
a) obtaining a [¹⁸F] source,
b) mixing the contents of said a first container with the contents of said second container with an anhydrous solvent to create a first exchange reaction mixture,
c) combining said [¹⁸F] source with said first exchange reaction mixture,
d) incubating said first exchange reaction mixture for at least 10 minutes,
e) adding water to said reaction mixture to quench said reaction mixture,
f) passing the quenched reaction mixture through said solid-phase extraction cartridge,
g) passing water through said solid-phase extraction cartridge, and
h) eluting the resulting [¹⁸F]N-heterocyclic carbene phosphorus(V) fluoride derivative from said solid-phase extraction cartridge with anhydrous solvent.

42. The kit of claim 41, wherein said anhydrous solvent is anhydrous acetonitrile.

43. The kit of claim 41, wherein said [¹⁸F] source comprises [¹⁸F]-tetra-n-butylammonium fluoride.

44. The kit of claim 41, wherein said incubating occurs at a temperature between room temperature and 100° C.
